(19) United States
(12) Patent Application Publication
(10) Pub. No.: US 2012/0135045 A1
(43) Pub. Date: May 31, 2012

(54) Antimicrobial Collagenous Constructs

(75) Inventors: Andrew J. Nixon, East Wareham, MA (US); Ginger A. Abraham-Freel, Hanover, MA (US); Patrick R. Bilbo, Sudbury, MA (US)

(73) Assignee: Organogenesis, Inc., Canton, MA (US)

(21) Appl. No.: 13/165,101

(22) Filed: Jun. 21, 2011

Related U.S. Application Data
(63) Continuation of application No. 12/090,631, filed on Oct. 14, 2008, now abandoned.
(60) Provisional application No. 60/728,414, filed on Oct. 18, 2005.

(51) Int. Cl. A61K 35/38; A61P 43/00; A61K 9/00; A61P 31/00; B82Y 5/00

(52) U.S. Cl. 424/400; 424/551; 977/810; 977/915

(57) ABSTRACT

Bioengineered collagen constructs with antimicrobial properties are provided. The bioengineered collagen constructs comprise a sheet-like layer of purified collagenous tissue matrix derived from a tissue source, such as the tunica submucosa of small intestine or a processed intestinal collagen layer derived from the tunica submucosa of small intestine, treated with an antimicrobial agent. The constructs are biocompatible. The present invention has a variety of applications, including wound dressing and surgical repair devices. Methods for treating a damaged or diseased soft tissue are provided. Methods for treating a wound in need of care and treatment are also disclosed.

### FIELD OF THE INVENTION

This invention is in the field of regenerative medicine and tissue engineering. The invention is directed to bioengineered constructs prepared from processed tissue material or matrix, derived from animal sources. The bioengineered constructs of the invention are prepared using methods that preserve biocompatibility, cell compatibility, strength, and bioremodelability of the processed tissue matrix. Antimicrobial properties are imparted to the bioengineered constructs, which are used for engraftment, implantation, tissue repair, wound repair and remodeling, or other use in a mammalian host.

### BRIEF DESCRIPTION OF THE BACKGROUND OF THE INVENTION

The field of regenerative medicine and tissue engineering combines the methods of engineering with the principles of life science to understand the structural and functional relationships in normal and pathological mammalian tissues. The goal of regenerative medicine and tissue engineering is the development and ultimate application of biological substitutes to restore, maintain, and improve tissue functions.

Collagen is the principal structural protein in the body and constitutes approximately one-third of the total body protein. It comprises most of the organic matter of the skin, tendons, bones and teeth, and occurs as fibrous inclusions in most other body structures. Some of the properties of collagen include high tensile strength; low antigenicity, due in part to masking of potential antigenic determinants by the helical structure; and low extensibility, semipermeability, and solubility. Furthermore, collagen is a natural substance for cell adhesion. These properties and others make collagen a suitable material for tissue engineering and manufacture of implantable biocompatible substitutes and bioremodelable prostheses.

Methods for obtaining collagenous tissue and tissue structures from explanted mammalian tissues and processes for constructing prosthesis from these tissues and tissue structures have been widely investigated for wound healing, surgical repair and for tissue or organ replacement. It is a continuing goal of researchers to develop bioengineered constructs that can successfully be used to raise the standard of healthcare that patients receive.

### SUMMARY OF THE INVENTION

Biologically-derived collagenous materials such as the intestinal submucosa are used in tissue repair or replacement and continue to be developed and improved. Novel bioengineered, bioremodelable constructs are now imparted with antimicrobial properties to improve their performance characteristics in regenerative medicine, including wound healing and tissue repair and replacement. Methods for mechanical and chemical processing of the proximal porcine jejunum to generate a single, acellular layer of intestinal collagen (ICL) derived from intestinal submucosa that can be used to form laminates with antimicrobial properties for healing, repair and replacement are disclosed. The processing removes cells and cellular debris while maintaining the native collagen tissue matrix structure. The resulting sheet of processed tissue matrix is used to prepare single layer and multi-layered laminated, crosslinked constructs with desired specifications. The efficacy of single layer wound dressing products, laminated multilayer patches for soft tissue repair as well as the use of entubated constructs as a vascular graft has been investigated. This intestine-derived processed tissue material provides the necessary physical support, while generating minimal adhesions, and is able to integrate into the surrounding native tissue and become infiltrated with host cells. In vivo bioremodeling does not compromise mechanical integrity of these constructs. Intrinsic and functional properties of the implant, such as the modulus of elasticity, suture retention and ultimate tensile strength are important parameters that can be manipulated for specific requirements by varying the number of layers and the crosslinking conditions. Antimicrobial qualities are now imparted to these constructs in order to control or lessen microbial activity at the treatment site where these constructs are used.

It is an object of the invention to provide a bioengineered collagen construct with antimicrobial properties comprising a sheet-like layer of purified collagenous tissue matrix derived from a tissue source, such as the tunica submucosa of small intestine or a processed intestinal collagen layer derived from the tunica submucosa of small intestine, treated with an antimicrobial agent. When the bioengineered collagen construct of the invention is used as a wound dressing to treat the wound of a mammalian patient, the construct is applied to the wound bed to substantially cover the wound such that the patient's own skin tissue is provided a moist environment in order to facilitate skin tissue regeneration while the antimicrobial agent in the construct controls or lessens microbial activity in the wound. The construct is biocompatible, meaning that the construct is non-cytotoxic, does not cause dermal desensitization and does not cause primary skin irritation.

In one embodiment, the wound dressing comprises a sheet of processed intestinal collagen derived from the tunica submucosa of small intestine having a thickness between about 0.05 to about 0.07 mm and an antimicrobial agent. Given the sheet-like geometry of the purified tissue matrix, it may be layered and then the layers chemically bonded together to provide a multilayered construct. Therefore, another embodiment is a construct comprising two, or more, layers of purified tissue matrix that have been bonded together and treated with an antimicrobial agent. The constructs of the invention may be meshed, perforated or fenestrated either for better conformation to a wound bed, better drainage of wound exudates, or both.

It is a further object in this aspect of the invention to treat a wound in need of care and treatment, particularly one in need of antimicrobial intervention and protection, where the wound comprises any one of the following types of wounds: partial and full thickness wounds, pressure ulcers, venous ulcers, diabetic ulcers, chronic vascular ulcers, tunneled/undermined wounds, surgical wounds, donor site wounds for autografts, post-Moh's surgery wounds, post-laser surgery wounds, wound dehiscence, trauma wounds, abrasions, lacerations, second-degree burns, skin tears or draining wounds.

It is another object of the invention to provide a surgical repair device, such as a patch or mesh, for the treatment and repair of soft tissues and organs, comprising two or more layers, such as between two and ten layers, of processed intestinal collagen derived from the tunica submucosa of small intestine that are bonded and crosslinked together to form a multilayer construct that is biocompatible and bioremodelable which, when implanted on the damaged or diseased soft tissue, undergoes controlled biodegradation occurring with adequate living cell replacement such that the original implanted prosthesis is remodeled by the patient's living cells. It is a further object in this aspect of the invention to provide a method for treating a damaged or diseased soft tissue in need of repair and antimicrobial intervention, comprising implantation of a prosthesis comprising two or more superimposed, chemically bonded layers of processed intestinal collagen derived from the tunica submucosa of small intestine, contacted with an antimicrobial agent, which, when implanted on the damaged or diseased soft tissue, undergoes controlled biodegradation occurring with adequate living cell replacement such that the original implanted prosthesis is remodeled by the patient's living cells. For example, the damaged or diseased soft tissue in need of repair are wounds, defects of the abdominal and thoracic wall, muscle flap reinforcement, rectal and vaginal prolapse, reconstruction of the pelvic floor, hernias, suture-line reinforcement and reconstructive procedures.

### DETAILED DESCRIPTION OF THE INVENTION

The invention is directed to a bioengineered collagen construct (e.g., prostheses, graft) comprising a sheet-like layer of purified collagenous tissue matrix, a processed tissue material, derived from native tissues, such as the processed intestinal collagen layer derived from tunica submucosa of small intestine, treated with an antimicrobial agent. The bioengineered collagen construct may be either a single layer of processed tissue matrix or may be a number of superimposed, bonded layers of processed tissue matrix. When the bioengineered collagen construct of the invention is used to treat a wound of a mammalian subject, it is applied to the wound bed to substantially cover the wound such that the subject's own skin tissue is provided both a moist environment in order to facilitate skin tissue regeneration and an antimicrobial composition to control or lessen microbial activity in the wound and at the wound periphery. When the bioengineered collagen construct of the invention is used as a surgical device, it is implanted to an implant site in a mammalian subject to serve as a functioning repair, augmentation, or replacement body part or tissue structure. The antimicrobial agent controls or lessens microbial activity in the wound or implant site by preventing adhesion and proliferation of bacteria on the construct. The antimicrobial constructs of the invention are biocompatible, meaning that the construct is non-cytotoxic, does not cause dermal desensitization and does not cause primary skin irritation.

The prostheses of the invention are also “bioremodelable,” meaning that it will undergo controlled biodegradation occurring concomitantly with remodeling and replacement with new endogenous matrix provided by the host's or patient's cells to create new tissue. Thus, a prosthesis of this invention when imparted with an antimicrobial agent and when used as a replacement tissue, has multiple properties. First, it functions as a substitute body part or wound covering. Second, while still functioning as a substitute body part, it functions as a remodeling template for the ingrowth of host cells. Third, it provides antimicrobial activity local to the treatment site.

The prosthetic material of this invention is a purified collagenous processed tissue matrix developed from mammalian-derived collagenous tissue that can be bonded to itself or another processed, purified tissue matrix and imparted with antimicrobial properties to form prosthesis for grafting or implantation to a site on a subject's body that requires treatment.

The invention includes methods for making tissue engineered prostheses from processed tissue material where the methods do not require adhesives, sutures, or staples to bond the layers together while maintaining the bioremodelability of the prostheses. The terms “processed tissue matrix” and “processed tissue material” mean native, normally cellular tissue that has been procured from an animal source, preferably a mammal, and mechanically cleaned of attendant tissues and chemically cleaned of cells, cellular debris and rendered substantially free of non-collagenous extracellular matrix components. The processed tissue matrix, while substantially free and purified of non-collagenous components, maintains much of its native matrix structure, organization, strength, and shape. Processed tissue material compositions for preparing the bioengineered grafts of the invention are derived from animal tissues comprising collagen with such collagenous tissue sources including, but not limited to: intestine, dermis, fascia lata, pericardium, dura mater, placenta and other flat or planar structured tissues that comprise a collagenous tissue matrix. The structure and geometry of these tissue matrices makes them able to be easily cleaned, manipulated, and assembled in a way to prepare the bioengineered grafts of the invention. Other suitable tissue sources with a similar flat structure, geometry and matrix composition may be identified, procured and processed by the skilled artisan in other animal sources in accordance with the invention.

One such processed tissue matrix composition for preparing the bioengineered grafts of the invention is an intestinal collagen layer derived from the tunica submucosa of small intestine. Suitable sources for small intestine are mammalian organisms such as human, cow, pig, sheep, dog, goat, or horse while small intestine of pig is a readily available source.

The prostheses of the invention may be prepared from the processed intestinal collagen layer (sometimes teemed “intestinal collagen layer” or “ICL”) which is a processed tissue material derived from the tunica submucosa of porcine small intestine. In one method for obtaining this intestinal collagen layer, the small intestine is harvested from a mammal and attendant mesenteric tissues are grossly dissected from the intestine. The tunica submucosa is separated, or delaminated, from the other layers of the small intestine by mechanically squeezing the raw intestinal material such as between opposing rollers similar to those in a sausage casing machine to remove the muscular layers (tunica muscularis) and the mucosa (tunica mucosa). As the tunica submucosa of the small intestine is harder and stiffer than the surrounding tissue, the rollers squeeze the softer components from the submucosa, resulting in a mechanically cleaned tissue matrix. In the examples that follow, the porcine small intestine was mechanically cleaned using a gut cleaning machine and then chemically cleaned in a series of solutions to yield a processed tissue matrix. This mechanically and chemically cleaned intestinal collagen layer derived from the tunica submucosa of small intestine is herein referred to as “ICL” and is one type of processed tissue matrix or material from which the antimicrobial constructs of the invention are prepared.

In composition, the processed ICL tissue material is acellular telopeptide Type I collagen, about 93% by weight dry, with less than about 5% dry weight glycoproteins, glycosaminoglycans, proteoglycans, lipids, non-collagenous proteins and nucleic acids such as DNA and RNA and is substantially free of cells and cellular debris. The processed ICL tissue material retains much of its matrix structure and strength. Importantly, the biocompatibility and bioremodelability of the tissue matrix is preserved in part by the cleaning process as it is free of bound detergent residues that would adversely affect the bioremodelability of the collagen. Additionally, the collagen molecules have retained their telopeptide regions as the tissue has not undergone treatment with enzymes during the cleaning process.

To obtain the processed tissue matrix, an appropriate animal and tissue source is determined. The tissue is processed both mechanically and chemically to remove attendant tissues and to remove non-collagenous components from the tissue to result in a processed tissue matrix. By way of example, ICL is one type of processed tissue matrix used in the production of the bioengineered graft prostheses of the invention. The methods described below are followed to process tissue to provide a processed tissue matrix and to fabricate a bioengineered graft prostheses comprising ICL and an antimicrobial agent.

To obtain porcine ICL, the tunica submucosa of porcine small intestine is used as a starting material for the bioengineered graft prosthesis of the invention. The small intestine of a pig is harvested, the attendant tissues are removed and then the intestine is mechanically cleaned using a gut cleaning machine which forcibly removes the fat, muscle and mucosal layers from the tunica submucosa using a combination of mechanical action and washing using water. The mechanical action can be described as a series of rollers that compress and strip away the successive layers from the tunica submucosa when the intact intestine is run between them. The tunica submucosa of the small intestine is comparatively harder and stiffer than the surrounding tissue, and the rollers squeeze the softer components from the submucosa. Other mechanical cleaning means in the art may be determined by the skilled artisan to include other physical manipulation such as scraping, squeezing, compressing and rubbing. The result of the mechanical cleaning is such that the submucosal layer of the intestine solely remains, a mechanically cleaned intestine.

After mechanical cleaning, a chemical cleaning treatment is employed to remove cell and matrix components from the mechanically cleaned intestine, preferably performed under aseptic conditions at room temperature. The mechanically cleaned intestine is cut lengthwise down the lumen and then cut into sections approximately 15 cm to 50 cm in length. Material is weighed and placed into containers at a ratio of about 100:1 v/v of solution to intestinal material. In the most preferred chemical cleaning treatment, such as the method disclosed in U.S. Pat. Nos. 5,993,844 and 6,599,690 to Abraham, the disclosures of which are incorporated herein, the collagenous tissue is contacted with an effective amount of chelating agent, such as ethylenediaminetetraacetic tetrasodium salt (EDTA) under alkaline conditions, such as by addition of sodium hydroxide (NaOH); followed by contact with an effective amount of acid where the acid contains a salt, such as hydrochloric acid (HCl) containing sodium chloride (NaCl); followed by contact with an effective amount of buffered salt solution such as 1 M sodium chloride (NaCl)/10 mM phosphate buffered saline (PBS); finally followed by a rinse step using water. Each treatment step is preferably carried out using a rotating or shaking platform to enhance the actions of the chemical and rinse solutions. The result of the cleaning processes is a processed intestinal collagen layer, or ICL, a mechanically and chemically cleaned processed tissue matrix derived from the tunica submucosa of small intestine. After rinsing, the ICL is then removed from the cleaning containers and gently compressed or blotted to remove excess water. At this point, the ICL may be stored frozen at −80° C., at 4° C. in sterile phosphate buffer, or dried until fabricated into a prosthesis. If stored dry, the ICL sheets are flattened on a surface such as a flat plate, preferably a porous plate or membrane, such as a polycarbonate membrane, and any lymphatic tags from the abluminal side of the material are removed using a scalpel, and the ICL sheets may be allowed to dry in a laminar flow hood at ambient room temperature and humidity.

The ICL is a planar sheet structure that can be used as a single layer material or to fabricate various types of constructs to be used as prostheses with the shape of the prostheses ultimately depending on their intended use. To form multilayer prostheses of the invention, ICL sheets are laminated using a method that continues to preserve the biocompatibility and bioremodelability of the processed matrix material but also is able to maintain its strength and structural characteristics for its performance as a replacement tissue. The processed tissue matrix derived from tissue retains the structural integrity of the native tissue matrix, that is, the collagenous matrix structure of the original tissue remains substantially intact and maintains physical properties so that it will exhibit many intrinsic and functional properties when implanted. When multilayer laminates of ICL are prepared, sheets of ICL are layered to contact another sheet. The area of contact is a bonding region where layers contact each other, whether the layers are directly superimposed on each other, or partially in contact or overlapping for the formation of more complex structures. In completed constructs, the bonding region must be able to withstand suturing and stretching while being handled in the clinic, during implantation and during the initial healing phase while functioning as a replacement body part. The bonding region must also maintain sufficient strength until the patient's cells populate and subsequently bioremodel the prosthesis to form a new tissue.

The processed tissue matrix is used as a single layer prosthesis or is formed into a multi-layered, bonded prosthesis of planar, tubular or complex shape. When the prostheses of the invention comprise two or more layers or processed tissue matrix, the layers are bonded by chemically crosslinking the layers together using a crosslinking agent. While chemical crosslinking is used to bond multiple layers of processed tissue matrix together, the degree of chemical crosslinking may be varied to modulate rates of bioremodeling throughout the prosthesis, that is the rates at which a prosthesis is both reabsorbed and replaced by host cells and tissue. In other words, the higher degree of crosslinking that is imparted to the prostheses of the invention, the slower the rate of bioremodeling the prostheses will undergo; conversely, the lower degree of crosslinking, the faster the rate of bioremodeling. Surgical indications dictate the extent and/or rate of bioremodeling required by the prosthesis. For example, when a single layer construct is used as a wound dressing, the prosthesis may or may not be chemically crosslinked. For example, as a surgical repair patch, or mesh, the prosthesis is a multilayer construct that has a low degree of crosslinking so that the prosthesis will bioremodel at a faster rate. For example, as a bladder sling to support a hypermobile bladder to prevent urinary incontinence, the prosthesis is a multilayer construct that has a high degree of crosslinking so that the prosthesis is not bioremodeled as fast, that is, it persists in substantially the same conformation in which it was implanted for a longer period of time.

The collagen matrix or construct, when in sheet form, generally has two opposing, large area surfaces. To treat the processed collagen matrix with an antimicrobial agent, antimicrobial agent is applied by contacting it to either side of the processed collagen matrix or it may be bound to both sides. Alternatively, the fibrous, absorptive qualities of the processed collagen matrix may be leveraged to apply the antimicrobial agent to the interior of the processed collagen material, as in the interstices of the fibrous processed tissue matrix, such as by immersing the collagen matrix in a solution containing the antimicrobial agent and allowing the solution to permeate the matrix through absorption. Another method for providing antimicrobial agent to the interior of a multilayer construct is to treat single layers of the processed tissue matrix and then laminating and bonding the layers together. For multilayer configurations, the methods include conducting the fabrication steps of treating the matrix sheets with an antimicrobial agent, layering the matrix sheets to form multiple layers and crosslinking the construct with a crosslinking agent in any order, including the following: treating the matrix sheets with an antimicrobial agent, layering the matrix sheets to form multiple layers, then crosslinking with a crosslinking agent; treating the matrix sheets with an antimicrobial agent, crosslinking with a crosslinking agent, then layering the matrix sheets to form multiple layers; crosslinking with a crosslinking agent, treating the matrix sheets with an antimicrobial agent, then layering the matrix sheets to form multiple layers; crosslinking with a crosslinking agent, layering the matrix sheets to form multiple layers, then treating the matrix sheets with an antimicrobial agent; layering the matrix sheets to form multiple layers, crosslinking with a crosslinking agent, then treating the matrix sheets with an antimicrobial agent; or, layering the matrix sheets to form multiple layers, treating the matrix sheets with an antimicrobial agent, then crosslinking with a crosslinking agent. In some cases, crosslinking the matrix may not be practical after coating with an antimicrobial agent as some of the antimicrobial agent may be washed out by the crosslinking agent.

Untreated and treated layers can be layered together in different arrangements to yield a prosthesis having localized antimicrobial agent. Methods for forming such multilayer configurations include layering processed tissue matrix sheets that have been treated with an antimicrobial agent and untreated matrix sheets together to form a multiple layer construct and crosslinking the layers together. For example, at least two matrix sheets may be layered, crosslinked, and antimicrobially treated to form a treated matrix construct, and then one or more untreated matrix sheets may be layered onto either or both of the upper or lower surfaces of the treated matrix construct, and then the resulting construct may be crosslinked to form a combination construct. Alternatively, at least two untreated matrix sheets may be layered and crosslinked to form an untreated matrix construct, and then one or more treated matrix sheets may be layered onto either or both of the upper or lower surfaces of the untreated matrix construct, and then the resulting construct may be crosslinked to form a combination matrix construct. In another example, treated and untreated matrix sheets may be layered alternately and then crosslinked to form a combination construct. In yet an other example, treated matrix sheets that have been treated with two different antimicrobial agents may be arranged in alternating or other order to provide a combination construct. In still yet another example, the orientation of the treated and untreated sheets to each other, or to treated or untreated matrix constructs in a combination matrix construct may be configured take advantage of the sidedness quality of the ICL material.

To treat only selected parts or areas of a prosthesis, portions of the surface of the material may be treated with an antimicrobial agent by masking portions of the surface to be treated such that the mask obstructs the antimicrobial agent from contacting the material while allowing other areas of the surface to be treated. Another way to localize an antimicrobial agent on a collagen matrix is to partially immerse the collagen material in a bath or reservoir such that only a portion of the collagen matrix contacts the antimicrobial agent and other portions remain free from contact with the antimicrobial treatment. Still another way to localize the antimicrobial agent on the surface of the material is to spray, or otherwise propel, the antimicrobial agent on one surface of the material while leaving the opposing surface untreated.

Single layer and multilayer constructs are treated with an antimicrobial agent to impart antimicrobial properties to the construct. At least one antimicrobial agent is applied to the constructs of the invention by contacting all or only part of the construct to the antimicrobial agent. Preferred antimicrobial agents include silver-based antimicrobial agents and chemical-based antimicrobial agents. An antibiotic agent may also be included in the composition. A combination of agents may be employed to treat the collagenous material to provide a wide spectrum of antimicrobial activity, for example, a silver-based antimicrobial agent and a chemical-based antimicrobial agent; a chemical-based antimicrobial agent and an antibiotic agent; a silver-based antimicrobial agent and an antibiotic agent; or a combination of all three types of agents.

Silver based antimicrobial agents may be selected to impart antimicrobial properties to prostheses comprising a processed tissue matrix. Silver may be applied to the collagen constructs in several forms. Silver based antimicrobial agents include silver or compounds containing silver that have some degree of antimicrobial activity and are compatible with both the collagen construct and the patient. Pure silver, also referred to as elemental or noble silver, is relatively chemically inactive and does not react with water or oxygen at normal temperatures and is not soluble in dilute acids and bases.

Ionic silver may also be used. It is believed that silver in ionic form has not yet produced microbial resistance and appears less likely to do so than other antimicrobial agents. While not wishing to be bound by theory, silver in this ionic form is effective against bacteria, yeast and fungi and extracellular viruses by directly affecting cell and cell wall respiration and transport as well as reproduction. Silver in other forms may also be used, including but not limited to: silver oxide; silver nitrate; silver sulfazidine (silver(I) sulfadiazine comprises an insoluble polymeric compound which releases silver ions slowly in its role as an antimicrobial and antifungal agent and may be used topically in the treatment of severe burns to prevent bacterial infection); silver-imidazolate; arglaes (AgKaPO₄); colloidal silver; silver crystals, such as silver nanocrystals, also termed “nanocrystalline silver,” is another way to deliver and sustain release of silver cation and its radicals to a wound or implant site.

Nanocrystalline silver (“nanosilver”) compositions are made according to several different methods. One method, a pulsed plasma method, produces nanocrystals with organized crystal lattice structure that lack atomic disorder but exhibit tight particle size distribution and morphology with a high degree of fidelity. The pulsed plasma process utilizes two conductive feedstock rods. The rods are fed into the reaction chamber filled with controlled gas, most likely an inert gas such as argon, at atmospheric pressure. The rods are connected to a high-powered, pulsed discharge power supply. Nanomaterial is synthesized by rapidly discharging power from the pulsed power supply across the feedstock rods. The high-powered discharge removes raw material to create a high temperature, high-pressure metal plasma. Due to using the unique gas dynamics, the plasma rapidly expands into the surrounding gas to create a homogeneous gas phase suspension of nanoparticles. The nanoparticles produced are continuously collected using a closed loop system. A blower recirculates the controlled gases carrying the particles to the collection system. This method produces nanocrystalline silver particles 10 nm to 100 nm in diameter depending on the process parameters. Generally particle sizes of between 15 nm to 40 nm, or 20 nm to 25 nm are selected for use in the invention.

Other methods for fabricating nanosilver compositions include those described in U.S. Pat. No. 6,719,987 to Burrell. These methods create crystals having a crystalline lattice characterized by atomic disorder. In the Burrell process, the material to be deposited is generated in the vapor phase, for example by evaporation or sputtering, and is transported into a large volume in which the temperature is controlled. Atoms of the material collide with atoms of the working gas atmosphere, lose energy and are rapidly condensed from the vapor phase onto a cold substrate, such as a liquid nitrogen cooled finger. Atomic disorder is created by conditions which limit diffusion such that sufficient atomic disorder is retained in the material. Deposition is conducted at low substrate temperatures for silver, from −10° to 100° C.; working gas pressures at higher than normal values are used; angles of incidence lower than about 30°; and higher deposition rates that create a higher than normal atom flux. Atomic disorder can also be achieved by the presence of different atoms or molecules in the metal matrix by a process called “doping” or by incorporating reactive gases (i.e., oxygen) into the chamber. According to this method, oxygen is a constituent in the process gas.

Chemical-based antimicrobial agents may be applied to collagen constructs to impart antimicrobial properties to the constructs. While not an exhaustive list, chemical antimicrobial agents may be selected from the following: Poly(hexamethylene biguanide) hydrochloride (PHMB); chlorhexadine gluconate; bis-amido polybiguanides such as those described in U.S. Pat. No. 6,316,669, the disclosure of which is incorporated herein; honey; benzalkonium chloride; triclosan (2, 4, 4′-tricloro-2′-hydroxydiphenylether); and silyl quarternary ammonium salt (octadecyl demethyl trimethoxysilyl propyl ammonium chloride).

In addition to an antimicrobial agent, the collagen construct may additionally comprise an antibiotic agent. An antibiotic agent is one that is produced by microorganisms to kill or inhibit the growth of other microorganisms. Generally, antibiotics are low molecular-weight (non-protein) molecules produced as secondary metabolites, mainly by microorganisms that live in the soil. Most of these microorganisms form some type of a spore or other dormant cell, and there is thought to be some relationship between antibiotic production and the processes of sporulation. Among the molds, the notable antibiotic producers are Penicillium and Cephalosporium, which are the main source of the beta-lactam antibiotics that include penicillin and related compounds. In the bacteria, the Actinomycetes, notably Streptomyces species, produce a variety of types of antibiotics including the aminoglycosides, for example, streptomycin, macrolides, for example, erythromycin, and the tetracyclines. Endospore-forming Bacillus species produce polypeptide antibiotics such as polymyxin and bacitracin. Antibiotics may have a cidal effect, which is a “killing” effect, or a static effect, meaning an “inhibitory” effect, on a range of microbes. The spectrum of action of an antibiotic agent is the range of bacteria or other microorganisms are affected by it. Antibiotics effective against prokaryotes which kill or inhibit a wide range of Gram-positive and Gram-negative bacteria are termed “broad spectrum”; those effective mainly against Gram-positive or Gram-negative bacteria are “narrow spectrum”; and those effective against a single organism or disease are “limited spectrum.” A preferred antibiotic compound to be used is a broad spectrum antibiotic. The antibiotic compounds may be provided to the collagen construct in combination, such as a combination of a narrow spectrum Gram-positive compound and a narrow spectrum Gram-negative compound; however, any combination of broad, narrow and limited spectrum range antibiotic may used.

Antibiotics for use in the invention include: beta-lactams (penicillins and cephalosporins), such as penicillin G, cephalothin; semisynthetic penicillin (which may also include clavulanic acid), such as ampicillin, amoxycillin and methicillin; monobactams, such as aztreonam; carboxypenems, such as imipenem; aminoglycosides, such as streptomycin; gentamicin; glycopeptides, such as vancomycin; lincomycins, such as clindamycin; macrolides, such as erythromycin; polypeptides, such as polymyxin; bacitracin; polyenes, such as amphotericin; nystatin; rifamycins, such as rifampicin; tetracyclines, such as tetracycline; semisynthetic tetracycline, such as doxycycline; and chloramphenicol.

The processed tissue matrix may be used as a single layer alone in a single layer prosthesis or used to fabricate prostheses having two or more layers. If used as a single layer, the antimicrobial agent is contacted to the processed tissue matrix to impart antimicrobial properties to the matrix. Either before or after contact with an antimicrobial agent, the processed tissue matrix may be crosslinked to control the bioremodeling and biodegradation rate of the material. In other words, the single layer antimicrobial constructs are: single layer and treated with an antimicrobial agent; single layer crosslinked with a crosslinking agent then treated with an antimicrobial agent; or single layer treated with an antimicrobial agent then crosslinked with a crosslinking agent.

Methods for making the multilayer prostheses comprising two or more layers of processed tissue matrix are described using ICL.

One embodiment of the invention is directed to flat sheet prostheses, and methods for making and using flat sheet prostheses, comprising of two or more layers of ICL bonded and crosslinked for use as an implantable biomaterial capable of being bioremodeled by a patient's cells. Due to the flat sheet structure of ICL, the prosthesis is easily fabricated to comprise any number of layers, preferably between 2 and 10 layers, more preferably between 2 and 6 layers, with the number of layers depending on the strength and bulk necessary for the final intended use of the construct. The ICL has structural matrix fibers that run in the same general direction. When layered, the layer orientations may be varied to leverage the general tissue fiber orientations in the processed tissue layers. The sheets may be layered so their fiber orientations are in parallel or at different angles. Layers may also be superimposed to form a construct with continuous layers across the area of the prosthesis. Alternatively, as the ultimate size of a superimposed arrangement is limited by the circumference of the intestine, the layers may be staggered, in collage arrangement to form a sheet construct with a surface area larger than the dimensions of the starting material but without continuous layers across the area of the prosthesis. Complex features may be introduced such as a conduit or network of conduit or channels running between the layers or traversing the layers, for example.

In the fabrication of a multilayer construct comprising ICL, an aseptic environment and sterile tools are preferably employed to maintain sterility of the construct when starting with sterile ICL material. To form a multilayer construct of ICL, a first sterile rigid support member, such as a rigid sheet of polycarbonate, is laid down in the sterile field of a laminar flow cabinet. If the ICL sheets are still not in a hydrated state from the mechanical and chemical cleaning processes, they are hydrated in aqueous solution, such as water or phosphate buffered saline. ICL sheets are blotted with sterile absorbent cloths to absorb excess water from the material. If not yet done, the ICL material is trimmed of any lymphatic tags on the serosal surface, from the abluminal side. A first sheet of trimmed ICL is laid on the polycarbonate sheet and is manually smoothed to the polycarbonate sheet to remove any air bubbles, folds, and creases. A second sheet of trimmed ICL is laid on the top of the first sheet, again manually removing any air bubbles, folds, and creases. This is repeated until the desired number of layers for a specific application is obtained, preferably between 2 and 10 layers.

The ICL has a sidedness quality from its native tubular state: an inner mucosal surface that faced the intestinal lumen in the native state and an opposite outer serosal surface that faced the ablumen. It has been found that these surfaces have characteristics that can affect post-operative performance of the prosthesis but can be leveraged for enhanced device performance. Currently with the use of synthetic devices, adhesion formation may necessitate the need for re-operation to release the adhesions from the surrounding tissue. In the formation of a pericardial patch or hernia repair prosthesis having two layers of ICL, it is preferred that the bonding region of the two layers is between the serosal surfaces as the mucosal surfaces have demonstrated to have an ability to resist postoperative adhesion formation after implantation. In other embodiments, it is preferred that one surface of the ICL patch prosthesis be non-adhesive, non-adherent and the other surface have an affinity for adhering to host tissue. In this case, the prosthesis will have one surface mucosal and the other surface scrosal. In still another embodiment, it is preferred that the opposing surfaces be able to create adhesions to grow together tissues that contact it on either side, thus the prosthesis will have serosal surfaces on both sides of the construct. Because only the two outer sheets potentially contact other body structures when implanted, the orientation of the internal layers, if the construct is comprised of more than two, is of lesser importance as they will likely not contribute to post-operative adhesion formation.

After layering the desired number of ICL sheets, the sheets are then bonded by dehydrating them together at their bonding regions, that is, where the sheets are in contact. While not wishing to be bound by theory, dehydration causes the collagen fibers of the ICL layers to come together when water is removed from between the fibers of the ICL matrix. The layers may be dehydrated either open-faced on the first support member or, between the first support member and a second support member, such as a second sheet of polycarbonate, placed before drying over the top layer of ICL and fastened to the first support member to keep all the layers in flat planar arrangement together with or without a small amount of pressure. To facilitate dehydration, the support member may be porous to allow air and moisture to pass through to the dehydrating layers. The layers may be dried in air, in a vacuum, or by chemical means such as by acetone or an alcohol such as ethyl alcohol or isopropyl alcohol. Dehydration may be done to room humidity, between about 10% Rh to about 20% Rh, or less; or about 10% to about 20% w/w moisture, or less. Dehydration may be easily performed by angling the frame holding the polycarbonate sheet and the ICL layers up to face the oncoming airflow of the laminar flow cabinet for at least about 1 hour up to 24 hours at ambient room temperature, approximately 20° C., and at room humidity.

While it is not necessary, in one embodiment, the dehydrated layers are rehydrated before crosslinking. The dehydrated layers of ICL are peeled off the porous support member together and are rehydrated in an aqueous rehydration agent, preferably water, by transferring them to a container containing aqueous rehydration agent for at least about 10 to about 15 minutes at a temperature between about 4° C. to about 20° C. to rehydrate the layers without separating or delaminating them.

The dehydrated, or dehydrated and rehydrated, bonded layers are then crosslinked together at the bonding region by contacting the layered ICL with a crosslinking agent, preferably a chemical crosslinking agent that preserves the bioremodelability of the ICL material. As mentioned above, the dehydration brings the collagen fibers in the matrices of adjacent ICL layers together and crosslinking those layers together forms chemical bonds between the components to bond the layers. Crosslinking the bonded prosthetic device also provides strength and durability to the device to improve handling properties. Various types of crosslinking agents are known in the art and can be used such as ribose and other sugars, oxidative agents and dehydrothermal (DHT) methods. A preferred crosslinking agent is 1-ethyl-3-(3-dimethylaminopropyl) carbodiimide hydrochloride (EDC). In another preferred method, sulfo-N-hydroxysuccinimide is added to the EDC crosslinking agent as described by Staros, J. V., Biochem. 21, 3950-3955, 1982. Besides chemical crosslinking agents, the layers may be bonded together with fibrin-based glues or medical grade adhesives such as polyurethane, vinyl acetate or polyepoxy. In the most preferred method, EDC is solubilized in water at a concentration preferably between about 0.1 mM to about 100 mM, more preferably between about 1.0 mM to about 10 mM, most preferably at about 1.0 mM. Besides water, phosphate buffered saline or (2-[N-morpholino]ethanesulfonic acid) (MES) buffer may be used to dissolve the EDC. Other agents may be added to the solution, such as acetone or an alcohol, up to 99% v/v in water, typically 50%, to make crosslinking more uniform and efficient. These agents remove water from the layers to bring the matrix fibers together to promote crosslinking between those fibers. The ratio of these agents to water in the crosslinking agent can be used to regulate crosslinking. EDC crosslinking solution is prepared immediately before use as EDC will lose its activity over time. To contact the crosslinking agent to the ICL, the hydrated, bonded ICL layers are transferred to a container such as a shallow pan and the crosslinking agent gently decanted to the pan ensuring that the ICL layers are both covered and free-floating and that no air bubbles are present under or within the layers of ICL constructs. The container is covered and the layers of ICL are allowed to crosslink for between about 4 to about 24 hours, more preferably between 8 to about 16 hours at a temperature between about 4° C. to about 20° C. Crosslinking can be regulated with temperature: at lower temperatures, crosslinking is more effective as the reaction is slowed; at higher temperatures, crosslinking is less effective as the EDC is less stable.

After crosslinking, the crosslinking agent is decanted and disposed of and the constructs are rinsed in the pan by contacting them with a rinse agent to remove residual crosslinking agent. A preferred rinse agent is water or other aqueous solution. Preferably, sufficient rinsing is achieved by contacting the chemically bonded construct three times with equal volumes of sterile water for about five minutes for each rinse. As described herein, antimicrobial properties are imparted to the constructs by contacting them with an antimicrobial agent either by contacting, or treating, each processed tissue matrix layer individually or by contacting, or treating, a multilayer intermediate construct. The method for treating processed tissue matrix in single or multilayer form with an antimicrobial agent will vary with the type of antimicrobial agent used but should be one that preserves the bioremodelable, biomechanical and biocompatible properties of the processed tissue matrix.

When nanocrystalline silver is selected as the antimicrobial agent, it may be applied to the collagen matrix material by contacting the collagen matrix material to the nanocrystalline silver. The antimicrobial agent may be applied by coating the processed matrix material by coating it by suspending the agent in solution. Nanocrystalline silver is dispersed in water, aqueous solution, or in a solvent to form a solution of dispersed nanocrystalline silver. The ICL is immersed in a tray with a volume of nanocrystalline silver solution such that, when the ICL is immersed, the nanocrystalline silver adheres to the ICL. The solution may also be agitated or stirred during immersion. The ICL is then removed from the solution and placed in an environment that allows the water or solvent to evaporate from the ICL with the bound silver to result in an ICL construct with a coating of nanocrystalline silver. Other methods for coating ICL with a dispersion containing nanocrystalline silver includes spraying and allowing the solvent to evaporate from the ICL.

When PHMB is selected as the antimicrobial agent, the PHMB is added to solution using 0.09%-0.5% in water v/v in which the processed tissue matrix is immersed so that the PHMB solution saturates the matrix. After a time sufficient for saturation of the solution into the processed tissue matrix, the matrix is removed from the solution and is allowed to dry so that the PHMB remains on the matrix when the solvent evaporates.

The processed tissue matrices and constructs may be treated or modified, either physically or chemically, prior to or after fabrication of a multi-layered, bonded graft prosthesis. Physical modifications such as shaping, conditioning by stretching and relaxing, or perforating, meshing or fenestrating the cleaned tissue matrices and constructs may be performed. Conditioning lessens the overall strain of the material while perforating, meshing or fenestrating provides for either better conformation to a wound bed or better passage and drainage of exudates, or both. Chemical modifications such as binding growth factors, selected extracellular matrix components, genetic material, and other agents that would affect bioremodeling and repair of the body part being treated, repaired, or replaced may also be performed.

Methods of physically modifying the tissue matrix and constructs of the invention may be determined by one of skill in the art when considering the requisite performance characteristics of the constructs. The constructs may be provided a pattern of perforations that communicate through the opposite sides of the construct by using a press with a die having needles, blades, or pegs arranged in pattern on the die face. The construct is placed on a platform and the die is pressed down such that the needles, blades, or pegs are pressed through the construct to the platform while puncturing the construct. A method for providing slits or a meshed pattern uses a skin meshing apparatus similar to those customarily used in autologous skin grafting procedures. One such apparatus is a Zimmer skin mesher. The constructs may be laser drilled to create micron sized pores through the completed prosthesis for aid in cell ingrowth using an excimer laser (e.g. at KrF or ArF wavelengths). The constructs may be perforated, fenestrated or laser drilled at any time during the process to make the prosthesis, but is preferably done before decontamination or terminal sterilization. For some indications it is preferred that the perforations or laser-drilled holes communicate through all layers of the prosthesis to aid in cell passage or fluid drainage. For other indications, it is preferred that they do not pass all the away across the layers so that the holes provide cell access to the interior of a multiplayer construct or to aid in neovascularization of the construct.

Other physical modifications to the single layer construct are perforations or fenestrations that communicate between both sides of the construct. Another physical modification to the single layer construct is to mesh the construct, which is, to provide an arranged pattern of slits to the construct so that it resembles a mesh. The slits are made in a pattern with a mesh ratio similar to that as provided by a skin mesher such as those made by Zimmer. Mesh ratios may be set at 1:1.5 or 2:1. To make a single layer ICL construct with antimicrobial properties, ICL is spread mucosal side down onto a smooth polycarbonate sheet, ensuring removal of creases, air bubbles and visual lymphatic tags. Spreading of the ICL over the polycarbonate sheet is performed to optimize the dimensions. Material is optionally adequately dried over its entire surface. At least one antimicrobial agent is then applied to the material. Material is optionally physically modified by meshing or perforating and then cut to size and packaged and finally sterilized per sterilization specifications. In an alternate embodiment, the antimicrobial treatment is applied to the material after the material has been physically modified with a mesh, perforations or fenestrations.

After the processed tissue matrix is prepared and coated with an antimicrobial agent, the constructs are trimmed to the desired size. For illustration, a usable size is about 6 inches square (approx. 15.2 cm×15.2 cm) but any size may be prepared and used for grafting to a patient.

The antimicrobial treated collagen matrix is then packaged in a container that is sealable for terminal sterilization, storage, and distribution. The antimicrobial treated collagen matrix may be packed in the container in a dry state or moist. Preferred packaging materials are compatible to the antimicrobial treatment, the collagen matrix and, if packaged in a moist state, any agents that keep the product moist. For constructs treated with light-sensitive antimicrobial agents, such as silver based antimicrobial agents, packaging materials that prevent or filter light passage are used to package the product to prevent reduction of antimicrobial activity and discoloration of the constructs.

Constructs are then terminally sterilized using means known in the art of medical device sterilization. A preferred method for sterilization is by contacting the constructs with sterile 0.1% peracetic acid (PA) treatment neutralized with a sufficient amount of 10 N sodium hydroxide (NaOH), according to U.S. Pat. No. 5,460,962, the disclosure of which is incorporated herein. Decontamination is performed in a container on a shaker platform, such as 1 L Nalge containers, for about 18±2 hours. Constructs are then rinsed by contacting them with three volumes of sterile water for 10 minutes each rinse. In a more preferred method, ICL constructs are sterilized using gamma irradiation between 25-37 kGy. Gamma irradiation significantly, but not detrimentally, decreases Young's modulus, ultimate tensile strength, and shrink temperature. The mechanical properties after gamma irradiation are still sufficient for use in a range of applications and gamma is a preferred means for sterilizing as it is widely used in the field of implantable medical devices. Dosimetry indicators are included with each sterilization run to verify that the dose is within the specified range. Constructs are packaged using a package material and design that is compatible with the composition of the construct and ensures sterility during storage. A preferred packaging means is a double-layer peelable package where the principal package is a heat-sealed, blister package comprised of a polyethylene terephthalate, glycol modified (PETG) tray with a paper surfaced foil lid that is enclosed in a secondary heat sealed pouch comprised of a polyethylenc/polyethylenctcrephthalate (PET) laminate. Together, both the principal and secondary package and the ICL construct contained therein are sterilized using gamma radiation.

The prostheses of this invention, may be flat, tubular, or of complex geometry. The shape of the formed prosthesis will be decided by its intended use. Thus, when forming the bonding layers of the prosthesis of this invention, the mold or plate support member can be fashioned to accommodate the desired shape. The flat multilayer prostheses can be implanted to repair, augment, or replace diseased or damaged organs, such as abdominal wall, pericardium, hernias, and various other organs and structures including, but not limited to, bone, periosteum, perichondrium, intervertebral disc, articular cartilage, dermis, bowel, ligaments, and tendons. In addition, the flat multilayer prostheses can be used as a vascular or intra-cardiac patch, or as a replacement heart valve.

Flat sheet prostheses, either in single or multiple layers, are used in wound healing to cover the wound of a subject to form a moisture barrier, to provide soothingess and comfort to the wound site and to provide antimicrobial activity as the wound site is stabilized to initiate healing.

Flat sheets may also be used for organ support, for example, to support prolapsed or hypermobile organs by using the sheet as a sling for the organs, such as bladder or uterus. Tubular prostheses may be used, for example, to replace cross sections of tubular organs such as vasculature, esophagus, trachea, intestine, and fallopian tubes. These organs have a basic tubular shape with an outer surface and an inner luminal surface. In addition, flat sheets and tubular structures can be formed together to form a complex structure to replace or augment cardiac or venous valves.

The ICL material used in the fabrication of the antimicrobial constructs of the invention are biocompatible. Biocompatibility testing has been performed on prostheses made from ICL in accordance with both Tripartite and ISO-10993 guidance for biological evaluation of medical devices. “Biocompatible” means that the prostheses are non-cytotoxic, hemocompatible, non-pyrogenic, endotoxin-free, non-genotoxic, non-antigenic, and do not elicit a dermal sensitization response, do not elicit a primary skin irritation response, do not case acute systemic toxicity, and do not elicit subchronic toxicity. These biocompatible qualities are discussed more in detail, below.

Test articles of constructs prepared from ICL showed no biological reactivity (Grade 0) or cytotoxicity observed in the L929 cells following the exposure period test article when using the test entitled “L929 Agar Overlay Test for Cytotoxicity In Vitro.” The observed cellular response to the positive control article (Grade 3) and the negative control article (Grade 0) confirmed the validity of the test system. Testing and evaluations were conducted according to USP guidelines. Prostheses of the invention are considered non-cytotoxic and meet the requirements of the L929 Agar Overlay Test for Cytotoxicity In Vitro.

Hemocompatibility (in vitro hemolysis, using the in vitro, modified ASTM extraction method test) testing of prostheses of the invention was conducted according to the modified ASTM extraction method. Under the conditions of the study, the mean hemolytic index for the device extract was 0% while positive and negative controls performed as anticipated. The results of the study indicate the prostheses of the invention are non-hemolytic and hemocompatible.

Prostheses of the invention were subjected to pyrogenicity testing following the current USP protocol for pyrogen testing in rabbits. Under conditions of the study, the total rise of rabbit temperatures during the observation period was within acceptable USP limits. Results confirmed that the prostheses of the invention are non-pyrogenic. The prostheses of the invention are endotoxin free, preferably to a level ≦0.06 EU/ml (per cm² of product). Endotoxin refers to a particular pyrogen that is part of the cell wall of gram-negative bacteria, which is shed by the bacteria and contaminates materials.

Prostheses of the invention do not elicit a dermal sensitization response. There are no reports in the literature that would indicate that the chemicals used to clean the porcine intestinal collagen elicit a sensitization response, or would modify the collagen to elicit a response. The results of sensitization testing on prostheses of the invention formed from chemically cleaned ICL indicate that the prostheses do not elicit a sensitization response.

Prostheses of the invention do no elicit a primary skin irritation response. The results of irritation testing on the chemically cleaned. ICL indicate that prostheses of the invention formed from chemically cleaned. ICL do not elicit a primary skin irritation response.

Acute systemic toxicity and intracutaneous toxicity testing was performed on chemically cleaned ICL used to prepare prostheses of the invention, the results of which demonstrated a lack of toxicity among the prostheses tested. Additionally, in animal implant studies there was no evidence that chemically cleaned porcine intestinal collagen caused acute systemic toxicity.

Subchronic toxicity testing of the prostheses of the invention containing porcine intestinal collagen confirmed lack of device subchronic toxicity.

There are no reports in the literature that would indicate that the chemicals used to clean the porcine intestinal collagen would affect the potential for genotoxicity, or would modify the collagen to elicit such a response. Genotoxicity testing of the prostheses of the invention containing porcine intestinal collagen confirmed lack of device genotoxicity.

The purpose of the chemical cleaning process for the porcine intestinal collagen used to prepare prostheses of the invention is to minimize antigenicity by removing cells, cell remnants, and non-collagenous and non-elastinous matrix components. Prostheses of the invention containing porcine intestinal collagen confirmed lack of device antigenicity, as confirmed by implant studies conducted with the chemically cleaned porcine intestinal collagen.

The ICL constructs of the invention are preferably rendered virally inactivated. In the manufacturing process, the efficacy of two chemical cleaning procedures, the NaOH/EDTA alkaline chelating solution (pH 11-12) and the HCL/NaCl acidic salt solution (pH 0-1), to inactivate four relevant and model viruses was tested. The model viruses were chosen based on the source porcine material, and to represent a wide range of physico-chemical properties (DNA, RNA, enveloped and non-enveloped viruses). The viruses included pseudorabies virus, bovine viral diarrhea virus, reovirus-3 and porcine parvovirus. The studies were conducted based on FDA and ICH guidance documents, including: CBER/FDA “Points to Consider in the Characterization of Cell Lines Used to Produce Biologicals (1993)”; ICH “Note for Guidance on Quality of Biotechnological Products: Viral Safety Evaluation of Biotechnology Products Derived from Cell Lines of Human or Animal Origin” (CPMP/ICH/295/95); and, CPMP Biotechnology Working Party “Note for Guidance on Virus Validation Studies: The Design, Contribution and Interpretation of Studies Validating the Inactivation and Removal of Viruses” (CPMP/BWP/268/95). The results of the study demonstrate that the cumulative viral inactivation of the two chemical cleaning steps is a clearance of greater than 10⁶ for all four model viruses. The data indicate that the chemical cleaning procedures are a robust and effective process that maintains the potential for inactivation of a large variety of viral agents.

The prostheses of the invention are bioremodelable. While functioning as a substitute body part or support, the prostheses of the invention also function as a bioremodelable matrix scaffold for the ingrowth of host cells. “Bioremodeling” is used herein to mean the production of endogenous structural collagen, vascularization, and cell repopulation by the ingrowth of host cells at a rate about equal to the rate of biodegradation, reforming and replacement of the matrix components of the implanted prosthesis by host cells and enzymes. The graft prostheses retain their structural characteristics while they are remodeled by the subjects in which they are implanted into all, or substantially all, host tissue, and as such, are functional as an analog of the tissue they repair or replace. In addition to these bioremodelable qualities, prostheses of the invention made from two or more layers of processed tissue matrix are prepared to incorporate desirable biomechanical properties.

Young's Modulus (MPa) is defined as the linear proportional constant between stress and strain. The Ultimate Tensile Strength (N/mm) is a measurement of the strength across the prosthesis. Both of these properties are a function of the number of layers of ICL in the prosthesis. When used as a load bearing or support device, it should be able to withstand the rigors of physical activity during the initial healing phase and throughout remodeling.

Lamination strength of the bonding regions is measured using a peel test. Immediately following surgical implantation, it is important that the layers not delaminate under physical stresses. In animal studies, no explanted materials showed any evidence of delamination. Before implantation, the adhesion strength between two opposing layers is about 8.1±2.1 N/mm for a 1 mM EDC crosslinked multilayer construct.

Shrink Temperature (° C.) is an indicator of the extent of matrix crosslinking. The higher the shrink temperature, the more crosslinked the matrix is in the material. A non-crosslinked, gamma-irradiated ICL has a shrink temperature of about 60.5±1.0.° C. In the preferred embodiment, EDC crosslinked prostheses will preferably have a shrink temperature between about 64.0±0.2° C. to about 72.5±1.1° C. for devices that are crosslinked in 1 mM EDC to about 100 mM EDC in 50% acetone, respectively.

The mechanical properties include mechanical integrity such that the prosthesis resists creep during bioremodeling, and additionally is pliable and suturable. The term “pliable” means good handling properties for ease in use in the clinic.

The term “suturable” means that the mechanical properties of the layer include suture retention that permits needles and suture materials to pass through the prosthesis material at the time of suturing of the prosthesis to sections of native tissue. During suturing, such prostheses must not tear as a result of the tensile forces applied to them by the suture, nor should they tear when the suture is knotted. Suturability of the prostheses, i.e., the ability of prostheses to resist tearing while being sutured, is related to the intrinsic mechanical strength of the prosthesis material, the thickness of the graft, the tension applied to the suture, and the rate at which the knot is pulled closed. Suture retention for a highly crosslinked flat 6-layer prosthesis crosslinked in 100 mM EDC and 50% acetone is about 6.7±1.6 N. Suture retention for a 2-layer prosthesis crosslinked in 1 mM EDC in water is about 3.7 N±0.5 N. The preferred lower suture retention strength is about 2N for a crosslinked flat 2-layer prosthesis as a surgeon's force in suturing is about 1.8 N.

As used herein, the term “non-creeping” means that the biomechanical properties of the prosthesis impart durability so that the prosthesis is not stretched, distended, or expanded beyond normal limits after implantation. As is described below, total stretch of the implanted prosthesis of this invention is within acceptable limits. The prosthesis of this invention acquires a resistance to stretching as a function of post-implantation cellular bioremodeling by replacement of structural collagen by host cells at a faster rate than the loss of mechanical strength of the implanted materials due from biodegradation and remodeling.

The processed tissue material of the present invention is “semi-pelmeable,” even though it has been layered and bonded. Semi-permeability permits the ingrowth of host cells for remodeling or for deposition of agents and components that would affect bioremodelability, cell ingrowth, adhesion prevention or promotion, or blood flow. The “non-porous” quality of the prosthesis prevents the passage of fluids intended to be retained by the implantation of the prosthesis. Conversely, pores, perforations, fenestrations, slits or a mesh may be formed in the prosthesis if a porous or perforated quality is required for an application of the prosthesis.

The mechanical integrity of the prosthesis of this invention is also in its ability to be draped or folded, as well as the ability to cut or trim the prosthesis obtaining a clean edge without delaminating or fraying the edges of the construct.

A sheet of processed intestinal collagen derived from the tunica submucosa of small intestine usually has a thickness between about 0.05 to about 0.07 mm. Given the sheet-like geometry of the purified tissue matrix, it may be layered and then chemically bonded together to provide a multilayered construct with greater thickness. The processed tissue matrix layers of the multilayered, bonded prosthetic device of the invention may be from the same collagen material, such as two or more layers of ICL, or from different collagen materials, such as one or more layers of ICL and one or more layers of fascia lata.

Constructs with antimicrobial properties may be used for the management of wounds including: partial and full thickness wounds, pressure ulcers, venous ulcers, diabetic ulcers, chronic vascular ulcers, tunneled/undermined wounds, surgical wounds (such as donor site wounds for autografts, post-Moh's surgery wounds, post-laser surgery wounds, wound dehiscence), trauma wounds (such as abrasions, lacerations, second-degree bums, and skin tears) and draining wounds. The wound dressing is a single-layer sheet or a multiple layer construct of mechanically and chemically cleaned porcine intestinal collagen, at least one antimicrobial agent, each processed tissue matrix sheet having about 0.05 to about 0.07 mm in thickness, containing fenestrations that communicate between both sides of the sheets. The product comprises primarily of Type I porcine collagen (about >95%) in its native form, with less than about 0.7% lipids and undetectable levels of glycosaminoglycans (about <0.6%) and DNA (about <0.1 ng/μl). The porcine intestinal collagen is substantially free of cells and cell remnants. The wound dressing of the invention may or may not crosslinked, but if crosslinked, crosslinked to a degree to regulate and control biodegradation, bioremodeling, or replacement of the dressing by a patient's cells. Physical modifications may be provided to the wound dressing to permit the passage fluids. The wound dressing provides a moist healing environment, a barrier to control and minimize bacterial contamination, and pain relief to a patient by covering sensory nerve terminals. An antimicrobial agent provides effective protection against microbial contamination in and around the wound site.

The wound dressing is applied to a patient with a wound in need of treatment. Before application, the wound area may be cleansed and debrided of scar tissue using standard wound dressing techniques. Preferably, debridement is to a degree where wound edges contain viable tissue. To apply the dressing of the invention, it is cut to the outline of the wound area. If the wound is larger than a single dressing piece, multiple pieces may be used and overlapped to provide coverage of the entire wound. If the dressing is in a dry state, it may be rehydrated using sterile saline or other isotonic solution. The edge of the dressing should be in contact with the intact tissue then smoothed into place to ensure that the dressing is in contact with the underlying wound bed. If excess exudate collects under the sheet, small openings can be cut in the sheet to allow the exudate to drain.

The antimicrobial wound dressing of the invention may be used as an interface between the wound and conventional, or secondary, wound dressing. Alternatively, the antimicrobial wound dressing of the invention may be used as an overlay to a skin replacement, such as an autograft, allograft or cultured skin construct. When used as an overlay, the wound dressing supplies a moist wound environment such that the cells from either the skin replacement may populate the wound bed for faster wound closure.

After application, an appropriate, non-adherent, secondary dressing is preferably applied in. order to maintain a moist wound environment. The optimum secondary dressing is determined by wound location, size, depth and user preference. The secondary dressing may be changed as needed to maintain a moist, clean wound area. Frequency of dressing changes will vary depending on the type, size and depth of the wound being treated, the volume of exudates produced and the type of dressing used. As healing occurs, the wound dressing may need to be replaced in which case additional applications of wound dressing may be performed until complete healing is achieved.

Other embodiments of the invention are directed to multilayer constructs imparted with antimicrobial properties. The prosthetic device of this invention has two or more superimposed collagen layers that are bonded together. As used herein, “bonded collagen layers” means composed of two or more layers of the same or different collagen material treated in a manner such that the layers are superimposed on each other and are sufficiently held together by self-lamination and chemical crosslinking.

More specifically, the prosthetic device is a surgical mesh or graft intended to be used for implantation to reinforce soft tissue including, but not limited to: defects of the abdominal and thoracic wall, muscle flap reinforcement, rectal and vaginal prolapse, reconstruction of the pelvic floor, hernias, bridging gaps in fascial defects, suture-line reinforcement and reconstructive procedures. One prosthetic mesh or graft of the invention comprises a five-layer sheet of porcine ICL and antimicrobial agent, about 0.20 mm to about 0.25 mm in thickness. The product consists primarily of Type T porcine collagen (about >95%) in its native form, with less than about 0.7% lipids and undetectable levels of glycosaminoglycans (about <0.6%) and DNA (about <0.1 ng/μl). The porcine intestinal collagen is substantially free of cells and cell remnants. The prosthesis is supplied sterile in sheet form in sizes ranging from 5×5 cm to 12×36 cm in double-layer peelable packaging. The prosthesis has a denaturation temperature of about 58±5° C.; a tensile strength of greater than 15N; a suture retention strength of greater than 2 N using a 2-0 braided silk suture; and, an endotoxin level of ≦0.06 EU/ml (per cm² of product). In particular, the prosthesis a flat sheet construct consisting of five layers of ICL and antimicrobial agent, bonded and crosslinked with 1 mM with 1-ethyl-3-(3-dimethylaminopropyl) carbodiimide hydrochloride (EDC) in water. To form this construct, a first sheet of ICL is spread mucosal side down. onto a smooth polycarbonate sheet; ensuring removal of creases, air bubbles and visual lymphatic tags. Spreading of the ICL is done to optimize dimensions. Three sheets of ICL (mucosal side down) are layered on top of the first, ensuring removal of creases, air bubbles and visual lymphatic tags when each sheet is layered. The fifth sheet should be layered with the mucosal side facing up, ensuring removal of creases and air bubbles. Visual lymphatic tags are removed prior to layering of this fifth sheet. The layers are dried together for 24±8 hours. The layers are now dried together and then are crosslinked in 1 mM EDC in water for 18±2 hours in 500 mL of crosslinking solution per 30 cm five layer sheet. Each product is rinsed with sterile water and is then cut to final size specifications while hydrated.

In an alternate embodiment, the prosthetic device is a surgical sling with at least one antimicrobial agent that is intended for implantation to reinforce and support soft tissues where weakness exists including but not limited to the following procedures: pubourethral support, prolapse repair (urethral, vaginal, rectal and colon), reconstruction of the pelvic floor, bladder support, sacrocolposuspension, reconstructive procedures and tissue repair. In another most preferred embodiment, the prosthetic device is a surgical sling comprised of three to five layers of bonded, crosslinked ICL treated with at least one antimicrobial agent. To fabricate a five layer device, ICL is spread mucosal side down onto a smooth polycarbonate sheet; ensuring removal of crcascs, air bubbles and visual lymphatic tags. Spreading of the ICL is done to optimize dimensions. A second, third, and fourth sheets of ICL (mucosal side down) are layered on top of the first, ensuring removal of creases, air bubbles and visual lymphatic tags when each sheet is layered. The fifth sheet is layered with the mucosal side facing up, ensuring removal of creases and air bubbles. Visual lymphatic tags should be removed prior to layering of this fifth sheet. (A three layer construct is made by a first sheet of ICL spread mucosal side down onto a smooth polycarbonate sheet; ensuring removal of creases, air bubbles and visual lymphatic tags; a second sheet of ICL (mucosal side down) layered on top of the first, and a third sheet layered on top of the second sheet with the mucosal side facing up.) The layers are dried for 24±8 hours and once dry, are crosslinked in 10 mM EDC in 90% acetone for 18±2 hours in 500 mL of crosslinking solution per 30 cm five layer sheet. Each bonded, crosslinked construct is rinsed with sterile water and is cut to final size specifications while hydrated. By providing pubourethral support, the sling may be used for the treatment of urinary incontinence resulting from urethral hypermobility or intrinsic sphincter deficiency. The surgical sling consists of a five-layer laminated sheet of porcine intestinal collagen, about 0.20 mm to about 0.25 mm in thickness, and an antimicrobial agent. The device is cross-linked with 1-ethyl-3-(3-dimethylaminopropyl) carbodiimide hydrochloride (EDC). The device consists primarily of Type I porcine collagen (about >95%) in its native form, with less than about 0.7% lipids and undetectable levels of glycosaminoglycans (about <0.6%) and DNA (about <0.1 Ng/μl). The porcine intestinal collagen is free of cells and cell remnants. The denaturation temperature (DSC) of the prosthesis is greater than about 63° C.; its tensile strength is greater than about 15N; its suture retention strength is greater than about 2N using a 2-0 braided silk suture; and the final endotoxin level is ≦0.06 EU/ml (per cm² of product). While the bioremodelable aspects of the sling can be varied and leveraged, the sling prosthesis of the invention is not a replacement body part, but an organ support device implanted as an assisting structure. It is preferred that the ICL layers of the sling be more highly crosslinked to reduce the bioremodelability of the sling. The sling prosthesis is a highly biocompatible, flexible, collagenous structure that, when implanted, maintains requisite structural support and strength while functioning as an organ support device.

In still another alternate embodiment, the prosthetic device is a dura repair patch that is intended for implantation to repair the dura mater, a tough membrane that protects the central nervous system. The dura repair device of the invention comprises of four layers of bonded, crosslinked ICL, and an antimicrobial agent. To fabricate a four layer device, ICL is spread mucosal side down onto a smooth polycarbonate sheet; ensuring removal of creases, air bubbles and visual lymphatic tags. Spreading of the ICL is done to optimize dimensions. A second and third sheets of ICL (mucosal side down) are layered on top of the first, ensuring removal of creases, air bubbles and visual lymphatic tags when each sheet is layered. The fourth sheet is layered with the mucosal side facing up, ensuring removal of creases and air bubbles. Visual lymphatic tags should be removed prior to layering of this fourth sheet. The layers are dried for 24±8 hours and once dry, are crosslinked in about 0.1 mM to about 1 mM EDC in 2-[N-morpholino]ethanesulfonic acid) (MES) buffer for 18±2 hours in 500 mL of crosslinking solution per 30 cm four layer sheet. Each bonded, crosslinked construct is rinsed with sterile water and is cut to final size specifications while hydrated. The dura repair device consists of a four-layer laminated sheet of porcine intestinal collagen, about 0.14 mm to about 0.21 mm in thickness. The device is cross-linked with 1-ethyl-3-(3-dimethylaminopropyl) carbodiimide hydrochloride (EDC). The device consists primarily of Type I porcine collagen (about >95%) in its native form, with less than about 0.7% lipids and undetectable levels of glycosaminoglycans (about <0.6%) and DNA (about <0.1 Ng/μl). The porcine intestinal collagen is free of cells and cell remnants. The denaturation temperature (DSC) of the prosthesis is greater than about 63° C.; it's tensile strength is greater than about 15N; it's suture retention strength is greater than about 2N using a 2-0 braided silk suture; and the final endotoxin level is ≦0.06 EU/ml (per cm² of product). The dura repair device is biocompatible and bioremodelable such that, when implanted into a patient in need of dura repair, it functions as a dura replacement while over time, is bioremodeled by host's cells that both degrade and replace the device such that a new host tissue replaces the device over time.

For instance, a multilayer construct of ICL is used to repair body wall structures. It may also be used as, for example, a pericardial patch, a myocardial patch, a vascular patch, a bladder wall patch, or a hernia repair device (as a tension free patch or a plug) or used as a sling to support hypermobile or prolapsed organs (rectocele, vault prolapse, cystocele). The multilayer construct is useful for treating connective tissue such as in rotator cuff or capsule repair. The multilayer construct is useful for dura repair to repair cranial defects after craniotomy procedures or to repair canal dura along the spinal cord. The material is useful in annular repair when the annular fibrosis is herniated (i.e., slipped disc) and is used as a plug in the hole created by the slipped disc or as a covering to the hole, or both. The material is useful in plastic surgery procedures such as mastopexy, abdominal surgery, and in facial plastic surgery (brow and cheek lifts). Both single and multilayer ICL materials may be used as a wound covering or dressing to assist in wound repair. Furthermore, it may also be implanted flat, rolled, or folded for tissue bulking and augmentation. A number of layers of ICL may be incorporated in the construct for bulking or strength indications. Before implantation, the layers may be further treated or coated with collagen or other extracellular matrix components, hyaluronic acid, heparin, growth factors, peptides, or cultured cells.

The following examples are provided to better explain the practice of the present invention and should not be interpreted in any way to limit the scope of the present invention. It will be appreciated that the device design in its composition, shape, and thickness is to be selected depending on the ultimate indication for the construct. Those skilled in the art will recognize that various modifications can be made to the methods described herein while not departing from the spirit and scope of the present invention.

### EXAMPLES

### Example 1

### Chemical Cleaning of Mechanically Cleaned Porcine Small Intestine

The small intestine of a pig was harvested and mechanically stripped, using a Bitterling gut cleaning machine (Nottingham, UK) which forcibly removes the fat, muscle and mucosal layers from the tunica submucosa using a combination of mechanical action and washing with water. The mechanical action can be described as a series of rollers that compress and strip away the successive layers from the tunica submucosa when the intact intestine is run between them. The tunica submucosa of the small intestine is comparatively harder and stiffer than the surrounding tissue, and the rollers squeeze the softer components from the submucosa. The result of the machine cleaning was such that the submucosal layer of the intestine solely remained.

The remainder of the procedure, chemical cleaning according to International PCT Application No. WO 98/49969 to Abraham, et al., the disclosure of which is incorporated herein by reference, was performed under aseptic conditions and at room temperature. The chemical solutions were all used at room temperature. The intestine was then cut lengthwise down the lumen and then cut into 15 cm sections. Material was weighed and placed into containers at a ratio of about 100:1 v/v of solution to intestinal material.

To each container containing intestine was added approximately 1 L solution of 0.22 μm (micron) filter sterilized 100 mM ethylenediaminetetraacetic tetrasodium salt (EDTA)/10 mM sodium hydroxide (NaOH) solution. Containers were then placed on a shaker table for about 18 hours at about 200 rpm. After shaking, the EDTA/NaOH solution was removed from each bottle.

To each container was then added approximately 1 L solution of 0.22 μm filter sterilized 1 M hydrochloric acid (HCl)/1 M sodium chloride (NaCl) solution. Containers were then placed on a shaker table for between about 6 to 8 hours at about 200 rpm. After shaking, the HCl/NaCl solution was removed from each container.

To each container was then added approximately 1 L solution of 0.22 μm filter sterilized 1 M sodium chloride (NaCl)/10 mM phosphate buffered saline (PBS). Containers were then placed on a shaker table for approximately 18 hours at 200 rpm. After shaking, the NaCl/PBS solution was removed from each container.

To each container was then added approximately 1 L solution of 0.22 μm filter sterilized 10 mM. PBS. Containers were then placed on a shaker table for about two hours at 200 rpm. After shaking, the phosphate buffered saline was then removed from each container.

Finally, to each container was then added approximately 1 L of 0.22 μm filter sterilized water. Containers were then placed on a shaker table for about one hour at 200 rpm. After shaking, the water was then removed from each container.

Processed ICL samples were cut and fixed for histological analyses. Hemotoxylin and eosin (H&E) and Masson's trichrome staining was performed on both cross-section and long-section samples of both control and treated tissues. Processed ICL tissue samples appeared free of cells and cellular debris while untreated control samples appeared normally and expectedly very cellular.

This single layer material of ICL may be used as a single layer or used to form bonded multilayer constructs, tubular constructs, or constructs with complex tubular and flat geometrical aspects.

### Example 2

### Method for Fabricating a Multilayer ICL Construct

ICL processed according to the method of Example 1 was used to form a multilayer construct having 2 layers of ICL. A sterile sheet of porous polycarbonate (pore size, manufacturer) was laid down in the sterile field of a laminar flow cabinet. ICL was blotted with sterile TEXWIPES (LYM-TECH Scientific, Chicopee, Mass.) to absorb excess water from the material. ICL material was trimmed of its lymphatic tags from the abluminal side and then into pieces about 6 inches in length (approx. 15.2 cm). A first sheet of trimmed ICL was laid on the polycarbonate sheet, mucosal side down, manually removing any air bubbles, folds, and creases. A second sheet of trimmed ICL was laid on the top facing, or abluminal side, of the first sheet with the abluminal side of the second sheet contacting the abluminal side of the first sheet, again manually removing any air bubbles, folds, and creases. The polycarbonate sheet with the ICL layers was angled up with the ICL layers facing the oncoming airflow of the laminar flow cabinet. The layers were allowed to dry for about 18±2 hours in the cabinet at room temperature, approximately 20° C. The dried layers of ICL were then peeled off the polycarbonate sheet together without separating or delaminating them and were transferred to a room temperature waterbath for about 15 minutes to hydrate the layers.

Chemical crosslinking solution of 100 mM EDC/50% Acetone was prepared immediately before crosslinking as EDC will lose its activity over time. The hydrated layers were then transferred to a shallow pan and the crosslinking agent gently decanted to the pan ensuring that the layers were both covered and free-floating and that no air bubbles were present under or within the constructs. The pan was covered and allowed to sit for about 18±2 hours in fume hood. The crosslinking solution was decanted and disposed. Constructs were rinsed in the pan three times with sterile water for about five minutes for each rinse. Using a scalpel and ruler, constructs were trimmed to the desired size.

Constructs were decontaminated with sterile 0.1% peracetic acid (PA) treatment neutralized with sodium hydroxide 10N NaOH according to U.S. Pat. No. 5,460,962, the disclosure of which is incorporated herein. Constructs were decontaminated in 1 L Nalge containers on a shaker platform for about 18±2 hours. Constructs were then rinsed with three volumes of sterile water for 10 minutes each rinse and PA activity was monitored by Minncare strip testing to ensure its removal from the constructs.

Constructs were then packaged in plastic bags using a vacuum sealer which were in turn placed in hermetic bags for gamma irradiation between 25.0 and 35.0 kGy.

### Example 3

### Zone of Inhibition Assay

The purpose of this Example is to illustrate how to test for an effective amount of an antimicrobial substance to single layer constructs prepared from processed tissue material derived from porcine intestinal submucosa (ICL) prepared to the method disclosed in Example 1 that will provide a microbial barrier as well as a microbial killer. Several antimicrobial substances were applied to ICL by either solubilizing or suspending the substances in solution, as follows:

1. 0.5% silver nitrate solution

2. 1% silver nitrate solution

3. 1-2% nanocrystalline silver solution in Isopropyl Alcohol (IPA)

4. 1-2% nanocrystallinc silver solution in 1×PBS

5. 1-2% polyhexamethylene biguanide (PHMB) in phosphate buffered saline solution

6. 1-2% polyhexamethylene biguanide (PHMB) in purified water

A chemically cleaned ICL was cut into 4 cm×6 cm pieces and each were placed, in both hydrated and dehydrated states, into approximately 100 mL of each solution and allowed to sit overnight. A treated ICL was then removed from the solutions and dried. To serve as a control condition, untreated ICL samples were used. Circular pieces were cut from each sample piece. Agar plates were streaked with bacteria. Samples were placed on plates and allowed to incubate for 24 hours for observation.

The nanocrystalline silver, (N.S.) solutions made in IPA dispersed completely and stayed in solution, while the N.S. in 1×PBS did not stay in solution and required shaking before adding the solution to the ICL. The TPA evaporated overnight and appeared even and uniform on both the hydrated and dehydrated piece of ICL, the dehydrated piece kept a more defined shape and was easier to handle. Both pieces were turned a charcoal black color. The side of ICL that faced down on the tray had a sidedness to it though it did not appear to affect the coating of the ICL or the effectiveness of the material in creating a large zone of inhibition, (ZOI). The N.S. products created the best ZOI and also created ZOI that did not allow bacteria growth into the 24-hour ZOI after 4 days as some of the other samples did. Although the silver nitrate samples exhibited ZOI that were smaller than the N.S. but larger than others, the reported toxicity of the silver nitrate does not necessarily make it the ideal additive to the ICL. The known antimicrobial properties of silver nitrate made it useful to compare N.S. against the silver nitrate, and the N.S. surpassed the effectiveness of the silver nitrate.

The PHMB solutions both appeared equal, while the ICL after soaking did not. The PBS solutions left the ICL with what appeared to be an uneven salt residue while the WFI solutions left the ICL with no apparent change. Both gave similar ZOI. They were smaller and did not create them in such a manner as with the N.S. ZOI.

Both nanocrystalline silver and PHMB were found to be effective antimicrobial agents that may be applied to ICL constructs as evidenced by their bactericidal effect in this assay.

### Example 4

### Treatment of 2-Layer Collagen Constructs Containing Antimicrobial Nanocrystalline Silver or PHMB

Laminated 2-layer ICL constructs were prepared (both laminated and crosslinked) and then treated with an antimicrobial agent to produce two-layer constructs with antimicrobial qualities.

Twelve 2-layer ICL constructs, each of approximately 9 cm×9 cm in size, were prepared according to Example 2. In their preparation, these ICL constructs were crosslinked using 10 mM EDC/0.1M MES [2-(N-morpholino)ethanesulfonic acid] (Pierce, Rockford, Ill.) buffer for 16-20 hours and were rinsed three times in sterile filtered water for 30 minutes. The prepared constructs were then treated with an antimicrobial agent.

Antimicrobial agents were prepared either as solutions or as dispersions. Five nanocrystalline silver dispersions were prepared by mixing 10, 1.0, 0.1, 0.01, 0.001 grams of nanocrystalline silver (Nanotechnologies (Ag-20) or equivalent, Austin, Tex.) in 1 L of dispersing agent, isopropyl alcohol (sterile filtered water is also an acceptable dispersing agent). A 0.2%, 0.1%, 0.02%, 0.002% PHMB solution was prepared by mixing Cosmocil CQ (20% PHMB solution, ArchChemicals, Inc., Norwalk, Conn.) with RODI/WFL

To coat the laminated and crosslinked constructs with an antimicrobial agent, the hydrated constructs were placed in 9.5 cm×9.5 cm trays. 50 mL of each agent were decanted into the trays, and the trays were placed on a shaker table. Coating times were 10 seconds, 1 hour, 3 hours and overnight (about 18 hours). Upon completion of the set coating times, the samples were allowed to dry on polycarbonate sheets in the sterile airflow of a laminar-flow biological safety cabinet to dry to 10-20% Rh.

The resulting constructs were 2-layer ICL constructs that had been laminated, crosslinked, and treated with an antimicrobial agent to impart antimicrobial qualities to the constructs.

### Example 5

### Treatment of Single-Layer Collagen Matrix with Antimicrobial Nanocrystalline Silver or PHMB Used to Fabricate 2-Laver Antimicrobial Constructs

Single-layer ICL constructs were prepared, crosslinked, treated with an antimicrobial agent and then laminated to form two-layer constructs to produce two-layer processed tissue matrix constructs with antimicrobial qualities. More antimicrobial agent was incorporated between the layers of ICL processed tissue matrix by laminating the constructs after the treatment with the antimicrobial agent.

The ICL was prepared according to Example 1, spread on polycarbonate sheets and lymph tags were removed. Pieces were cut in sizes of approximately 10 cm×9 cm. Each piece of ICL was then crosslinked in 10 mM EDC/0.1M MES [2-(N-morpholino)ethanesulfonic acid] (Pierce , Rockford, Ill.) buffer, 2 liters per 26 pieces and agitated on a shaker table set to 4, for 16-20 hours and then rinsed three times in minimum of 2 L in sterile filtered water for a minimum of 20 minutes per rinse.

Antimicrobial agents were prepared either as solutions or as dispersions. Three nanocrystalline silver dispersions were prepared by mixing 1.0, 0.1, 0.01 grams of a nanocrystalline silver composition, (Nanotechnologies (Ag-20) or equivalent, Austin, Tex.) in 1 L of dispersing agent, such as isopropyl alcohol (sterile filtered water is also an acceptable dispersing agent). 0.2%, 0.1%, 0.02% PHMB solutions were prepared by mixing Cosmocil CQ (20% PHMB solution, ArchChemicals, Inc. Norwalk, Conn.) with RODI/WFI.

To coat ICL with an antimicrobial agent, pieces were placed in square 125 ml sterile containers (Nalgene) with four pieces per container. 100 mL of solution or dispersion containing antimicrobial agent were decanted into each container to immerse the constructs. ICL remained immersed for 3-6 hours while the containers were agitated on a rotating shaker platform.

The antimicrobial-treated ICL was then layered to form two-layer constructs by placing one treated piece of ICL flat against a polycarbonate sheet. A second treated piece of ICL was then placed directly on top and spread over the first so that no air bubbles were present between the layers. The polycarbonate sheets with the constructs were then placed in the sterile airflow of a laminar-flow biological safety cabinet to dry to 10-20% Rh for a minimum of 12 hours.

The resulting constructs were 2-layer ICL constructs that had been crosslinked and treated with an antimicrobial agent so as to impart antimicrobial qualities to the constructs, and then laminated so as to incorporate antimicrobial agent not just on the outer surfaces of the constructs but also between the layers of the constructs.

### Example 6

### Treatment of 2-Layer Collagen Constructs Containing Antimicrobial Nanocrystalline Silver

Laminated 2-layer ICL constructs were prepared (both laminated and crosslinked) and then treated with an antimicrobial agent to produce two-layer constructs with antimicrobial properties.

Twelve 2-layers ICL constructs, each approximately 35-40 cm×9 cm in size, were prepared according to Example 2. During their preparation, these ICL constructs were crosslinked using 10 mM EDC/0.1M MES [2-(N-morpholino)ethanesulfonic acid] (Pierce, Rockford, Ill.) buffer in water for 16-20 hours and were rinsed three times in sterile filtered water for 30 minutes. The prepared constructs were then treated with an antimicrobial agent.

Antimicrobial agents were prepared as dispersions. Three nanocrystalline silver dispersions were prepared by mixing 10.0, 5.0, 1.0 grams of nanocrystalline silver (Nanotechnologies (Ag-20) or equivalent, Austin, Tex.) in 1 L of isopropyl alcohol as dispersing agent for the nanocrystalline silver (sterile filtered water is also an acceptable dispersing agent).

To coat the constructs with an antimicrobial agent, the constructs were placed in 1 L square containers (Nalgene) with four constructs per container. 200 mL of solution or dispersion containing an antimicrobial agent were decanted into each container to immerse the constructs. The constructs remained immersed for 3-6 hours while the containers were agitated on their side on a rotating shaker platform. Constructs that had been contacted with the nanocrystalline silver dispersions were rinsed once in 1 L of sterile filtered water. Antimicrobial-treated constructs were then laid flat on polycarbonate sheets in the sterile airflow of a laminar-flow biological safety cabinet to dry to 10-20% Rh for a minimum of 12 hours.

The resulting constructs were 2-layer ICL constructs that had been laminated, crosslinked and treated with an antimicrobial agent so as to impart antimicrobial qualities to the constructs.

### Example 7

### Treatment of Single-Layer Collagen Matrix with Antimicrobial Nanocrystalline Silver Used to Fabricate 2-Layer Antimicrobial Constructs

Crosslinked single-layer ICL constructs were prepared, crosslinked, treated with an antimicrobial agent and then laminated to form two-layer constructs to produce two-layer constructs with antimicrobial qualities. By laminating the constructs following the treatment with the antimicrobial agent, it is possible to incorporate more antimicrobial agent into the constructs by coating the outer surfaces and was present between the layers of the construct.

ICL was prepared according to Example 1. ICL was then spread on polycarbonate sheets 35-40 cm long×9 cm width and lymph tags were removed. Each piece of ICL was then crosslinked in 10mM EDC/0.1M MES [2-(N-morpholino)ethanesulfonic acid] (Pierce, Rockford, Ill.) buffer, 3 liters per 30 pieces, agitated on a shaker table set to 4, for 16-20 hours and then rinsed three times in 3-5 L in sterile filtered water for 30 minutes per each rinse.

Antimicrobial agents were prepared as dispersions. Four nanocrystalline silver dispersions were prepared by mixing 10.0, 5.0, 1.0 grams of nanocrystalline silver (Nanotechnologies (Ag-20) or equivalent, Austin, Tex.) in 1 L of a dispersing agent as isopropyl alcohol. A 5.0 g nanocrystalline in 1 L of RODI was also prepared.

To coat ICL with an antimicrobial agent, ICL pieces were placed in 250 mL sterile containers (Nalgene) with four pieces per container. 200 mL of solution or dispersion containing an antimicrobial agent were decanted into each container to immerse the constructs. ICL remained immersed for 3-6 hours while the containers were agitated on a rotating shaker platform. ICL that had been contacted with the nanocrystalline silver dispersion were rinsed once in 1 L of sterile filtered water.

Each antimicrobial-treated ICL was then layered to form two-layer constructs by placing one treated piece of ICL flat against a polycarbonate sheet. A second treated piece of ICL was then placed directly on top and spread over the first so that no air bubbles were present between the layers. The polycarbonate sheets with the constructs were then placed in the sterile airflow of a laminar-flow biological safety cabinet to dry to 10-20% Rh for a minimum of 12 hours.

The resulting constructs were 2-layer ICL constructs that had been crosslinked and treated with an antimicrobial agent so as to impart antimicrobial qualities to the constructs, and then laminated so as to incorporate more antimicrobial agent between the layers of the construct.

### Example 8

### Coating Post Cross-Linking and Pre-Lamination

Crosslinked single-layer ICL constructs were prepared and crosslinked, then treated with an antimicrobial agent and then laminated to form two-layer constructs to produce two-layer constructs with antimicrobial qualities. By laminating the constructs after the treatment with the antimicrobial agent, more antimicrobial agent was incorporated between the layers of ICL.

ICL was prepared according to Example 1. Pieces of ICL were then spread on polycarbonate sheets and lymph tags were removed and were about 35-40 cm long×9 cm wide. Each piece of ICL was then crosslinked in 10 mM EDC/0.1 M MES buffer [2-(N-morpholino)ethanesulfonic acid] (Pierce, Rockford, Ill.), 3 liters per 30 pieces, agitated on a shaker table set to 4, for 16-20 hours and then rinsed three times in 3-5 L in sterile filtered water for 30 minutes per rinse.

Antimicrobial agents were prepared either as solutions or as dispersions. A nanocrystalline silver dispersion was prepared by mixing 5.0 grams of nanocrystalline silver (Nanotechnologies (Ag-20) or equivalent, Austin, Tex.) in 1 L of dispersing agent, RODI. A 0.1% PHMB solution was prepared by mixing 5.0 mL of Cosmocil CQ (20% PHMB solution) per 1000 mL of RODI/WFI.

To coat ICL with the PHMB agent, 28-30 ICL pieces were placed in a 5L clean Pyrex glass bottle. 3000 mL of 0.1% PHMB solution was added. ICL remained immersed for 3-6 hours while the containers were agitated on a rotating shaker platform.

To coat ICL with the nanocrystalline silver dispersion, 200 mL were added into 250 mL sterile containers (Nalgene). Four pieces of ICL were put into each container and agitated for 3-6 hours. ICL that had been contacted with the nanocrystalline silver dispersion were rinsed once in 250 mL of RODI for 15 minutes and once in 500 mL of RODI for minimum of 5 minutes until laminated. ICL that had been contacted with PHMB solution were not rinsed.

The antimicrobial-treated ICL was then layered to form two-layer constructs by placing one treated piece of ICL flat against a polycarbonate sheet. A second treated piece of ICL was then placed directly on top and spread over the first so that no air bubbles were present between the layers. The polycarbonate sheets with the constructs were then placed in the sterile airflow of a laminar-flow biological safety cabinet to dry to 10-20% Rh for a minimum of 12 hours.

The resulting constructs were 2-layer ICL constructs that had been crosslinked and treated with an antimicrobial agent so as to impart antimicrobial qualities to the constructs, and then laminated so as to incorporate more antimicrobial agent between the layers of the construct.

### Example 9

### Evaluation of Three Antimicrobial Dressings on the Proliferation of Methicillin Resistant Staphylococcus aureus (MRSA) in a Partial Thickness Wound Model

The antibacterial activity of three antimicrobial dressings on partial thickness wounds that were colonized with Methicillin resistant Staphylococcus aureus (MRSA) was examined.

One pig was used as the experimental research animal since their skin is morphologically similar to human skin. The pig weighed. approximately 25-30 kg and was kept in house for two weeks prior to initiating the experiment. This pig was fed a basal diet ad libitum and housed alone in the animal facility (meeting USDA compliance) with controlled temperature (19-21° C.) and lights (12 h/12 h LD). The pig was anesthetized with Telazol (5 mg/kg), Xylazine (2 mg/kg), Atropine (0.05 mg/kg) I.M. and inhalation of an isofluorane and oxygen combination. The hair on the back of the pig was clipped with standard animal clippers. Skin on both sides of the back of the pig was prepared by washing with a non-antibiotic soap (Neutrogena®) and sterile water. The animal was blotted dry with sterile gauze. Thirty six (36) partial thickness wounds (10×7×0.3mm) were made on the dorsal skin by using a specialized electrokeratome. The wounds were then inoculated with Methicillin resistant Staphylococcus aureus ATCC 33593.

To prepare the bacterial inoculation, a fresh culture of pathogenic isolate obtained directly from American Type Culture Collection (ATCC), Rockville, Md., was used. The inoculum was Methicillin resistant Staphylococcus aureus ATCC 33593. The freeze-dried bacteria culture was recovered per ATCC standard recovering protocol. All inoculum suspensions were made by scraping the overnight growth from a culture plate into 5 ml of normal saline until the turbidity of the suspension is equivalent to that of a MacFarland #8 Turbidity Standard. This will result in a suspension concentration of approximately 10⁸ colony forming units/ml (CFU/ml). The 10⁸ suspension was serially diluted to make an inoculum suspension with a concentration of approximately 10⁶ CFU/ml. A small amount of the inoculum suspension was plated onto culture media to quantify the exact concentration of viable organisms. The inoculum suspension was used directly to inoculate each wound site. A 0.025 ml (25 μl) aliquot of the suspension will be deposited into a sterile glass cylinder (22 mm diameter) in the center of each wound. The suspension was lightly scrubbed into each test site for ten seconds using a sterile Teflon spatula and let dry for 3 minutes. Within 10 minutes of inoculation, all inoculated wounds were covered with a polyurethane film dressing for 24 hours before the initiation of the treatment in order to give the bacteria time to colonize the wounds.

Three additional wounds were created and inoculated to obtain a baseline CPU/ml before initiation of the treatments: Inoculum: Log 4.24 CFU/mL; Baseline Counts: Log 5.38 CFU/mL. Six wounds were assigned to various treatment groups as seen by the below experimental design. The animal subject was monitored daily for any observable signs of pain or discomfort. In order to help minimize possible discomfort, an analgesic (Duragesic—fentanyl transdermal system that elutes at 25 μg/hr) was used during the entire experiment.

1) Dressing A was 2-layer PHMB treated construct made according to Example 8

2) Dressing B was 2-layer nanocrystalline silver treated construct made according to Example 8

3) Dressing C was 1-layer nanocrystalline silver treated construct made according to Example 8

4) Control (petroleum gauze or polyurethane film only)

5) Positive Control (competitor antimicrobial dressing or Bactroban ointment)

6) Untreated, air exposed controlAll treatment groups except untreated were covered with polyurethane film (as discussed with sponsor). Treatment groups were randomly assigned to either area 1, 2, 3, 4, 5 or 6. A total of 3 wounds per treatment group per day were assessed.

Three wounds were cultured from each treatment group on 24 and 72 hours post treatment. At each sampling time, sites were cultured quantitatively. Each site was cultured only once. The area was encompassed by a sterile glass cylinder (22 mm outside diameter) held in place by two handles. One ml of scrub solution was pipetted into the glass cylinder and the site was scrubbed with a sterile Teflon spatula for 30 seconds. Serial dilutions were made and scrub solutions were quantified using the Spiral Plater System, which deposits a small-defined amount (40 μl) of suspension over the surface of a rotating agar plate.

MRSA was grown on selective media prepared with Mueller Hinton agar, 4% NaCl and 6 micrograms of oxacillin/ml. Oxacillin was used instead of methicillin because it is more stable. After the incubation period (24 hrs), colonies on the plates were counted and the colony forming units per mL (CFU/ml) calculated.

Results from this study are presented in Table 1. Results show that the 2-layer PHMB, 2-layer nanocrystalline silver, and 1-layer nanocrystalline silver were effective in eradicating bacteria from the wounds.

TABLE 1
MRSA recovery (Log CFU/ML)

| | | | Average | |
|---|---|---|---|---|
| Assessment | | | (Log | |
| Time | Treatment | Log CFU/mL | CFU/mL) | STDEV |
| 24 hours | PHMB 2 Layer | 5.13 | 5 | 0.63 |
| | | 4.31 | | |
| | | 5.56 | | |
| | 2 Layer Nano Silver | 5.46 | 5.99 | 0.57 |
| | | 6.6 | | |
| | | 5.93 | | |
| | 1 Layer Nano Silver | 3.97 | 4.22 | 0.48 |
| | | 3.92 | | |
| | | 4.78 | | |
| | Acticoat | 5.6 | 5.27 | 0.89 |
| | | 5.97 | | |
| | | 4.26 | | |
| | Bioclusive | 6.27 | 6.61 | 0.36 |
| | | 6.56 | | |
| | | 7 | | |
| | Untreated Air | 6.58 | 6.41 | 0.14 |
| | | 6.33 | | |
| | | 6.33 | | |
| 72 hours | PHMB 2 Layer | 3.08 | 3.17 | 0.92 |
| | | 4.15 | | |
| | | 2.3 | | |
| | 2 Layer Nano Silver | 5.45 | 5.61 | 0.38 |
| | | 6.05 | | |
| | | 5.34 | | |
| | 1 Layer Nano Silver | 5.04 | 5.08 | 0.17 |
| | | 5.27 | | |
| | | 4.93 | | |
| | Acticoat | 5.57 | 5.93 | 0.480 |
| | | 6.48 | | |
| | | 5.76 | | |
| | Bioclusive | 5.81 | 5.81 | 0.25 |
| | | 6.06 | | |
| | | 5.56 | | |
| | Untreated Air | 5.26 | 4.67 | 0.54 |
| | | 4.19 | | |
| | | 4.56 | | |

### Example 10

### Mechanical Testing Techniques and Properties of Multilayer ICL Prostheses

Preferred embodiments of multilayer ICL patch constructs were tested. Constructs of 2, 4, and 6 layers of ICL crosslinked with 100 mM EDC in 50% Acetone (100/50) and 6 layer constructs with crosslinked with 7 mM EDC/90% acetone v/v in water (7/90) and 1 mM EDC in water (1/0) were evaluated along a number of measures. Results are summarized in Table 2.

Tensile failure testing was performed using a servohydraullic MTS testing system with TestStar-SX software. Strips 1.25 cm in width were pulled to failure in uniaxial tension at a constant strain rate of 0.013 s⁻¹. The slope of the linear region (E_{Y}) and the ultimate tensile strength (UTS) were calculated from the stress strain curves.

The adhesion strength between the layers was tested using a standard protocol for the testing of adhesives (ASTM D1876-95). The adhesion strength is the average force required to peel apart two layers of laminated ICL at a constant velocity of 0.5 cm/sec.

A differential scanning calorimeter was used to measure the heat flow to and from a sample under thermally controlled conditions. The shrink temperature was defined as the onset temperature of the denaturation peak in the temperature-energy plot. Suture retention was not performed on 2 or 4 layer constructs cross-linked in 100 mM EDC and 50% acetone since the suture retention (3.7N±0.5N) for a 2 layer construct cross-linked in 1 mM EDC and no acetone (much less cross-linked) was well above the 2 N minimum specification. Lamination strength between ICL layers and shrinkage temperature are dependent on the crosslinking concentration and the addition of acetone rather than the number of layers in a construct.

TABLE 2
Mechanical Properties of Multilayer ICL Constructs

| | 2 Layer | 4 Layer | 6 Layer | 6 Layer | 6 Layer |
|---|---|---|---|---|---|
| | 100 mM | 100 mM | 100 mM | 70 mM | 1 mM EDC |
| Mechanical | EDC/50% | EDC/50% | EDC/50% | EDC/90% | in Water (n [symbol] |
| Analysis | Acetone | Acetone | Acetone | Acetone | acetone) |
| Ultimate Tensile | 0.6 ± 0.1 | 3.1 ± 0.2 | 2.0 ± 0.2 | 2.7 ± 0.2 | 1.3 ± 0.4 |
| Strength (N/mm) | | | | | |
| Young's Modulus (MPa) | 38.0 ± 5.8 | 49.5 ± 4.0 | 35.9 ± 2.6 | 43.0 ± 1.2 | 14.5 ± 7.8 |
| Lamination | 39.7 ± 6.1 | | | 63.1 ± 24.4 | 8.1 ± 2.1 |
| Strength (N/m) | | | | | |
| Suture Retention (N) | not tested | not tested | 6.6 ± 1.6 | 10.6 ± 2.2 | 10.9 ± 2.8 |
| Shrink | 72.5 ± 1.1 | | | 69.5 ± 0.1 | 64.0 ± 0.2 |
| Temperature (° C.) | | | | | |

[symbol]

indicates data missing or illegible when filed

### Example 11

### Method for Treating a Wound with an Antimicrobial Treated Construct

Either a single sheet layer of ICL from Example 1 or a bonded multilayer sheet construct of ICL formed by the method of Example 2 is used to treat a full-thickness skin wound. The sheet is meshed or fenestrated to create small openings to allow for seepage of wound exudate.

Skin wounds including second degree burns, lacerations, tears and abrasions; surgical excision wounds from removal of cancerous growths or autograft skin donor sites; and skin ulcers such as venous, diabetic, pressure (bed sores), and other chronic ulcers are managed using ICL in single or multilayer form. The collagenous ICL matrix protects the wound bed while maintaining moisture and allowing drainage from the wound. Before the ICL is applied to the wound, the wound bed is prepared for its application.

Patients with burn wounds requiring grafting are selected. ICL is placed either directly on the excised wound bed or over meshed autograft unexpanded or expanded at a ratio of 2:1 or more. Test sites (ICL) and control sites (autograft), when used, are of the same mesh ratio. The burned wound sites to be grafted are prepared, such as by debridement, prior treatment according to standard practice so that the burned skin area was completely excised. Excised beds appear clean and clinically uninfected.

Patients undergoing surgical excision are locally anesthetized. The pre-operative area is cleansed with an anti-microbial/antiseptic skin cleanser (Hibiclens®) and rinsed with normal saline. Deep partial thickness wounds are made in the skin and the skin is grafted elsewhere unless it is cancerous. ICL is applied to the wound bed and sterile bandages are applied.

In either wound case, appropriate post-operative care is provided to the patient in examination, cleaning, changing bandages, etc. of the treated wounds. A complete record of the condition of the treated sites is maintained to document all procedures, necessary medications, frequency of dressing changes and any observations made. The wound beds remain protected from the external environment and moist to aid in wound management and healing.

The wound dressing was tested in an animal model. The wound dressing construct of the invention is either a single or multilayer sheet construct made from ICL formed by the methods of Examples 1 and 2. A rat full-thickness wound healing model (a commonly used model for wound dressing products) was used to assess the performance of a wound dressing construct made from a single layer material of ICL. A total of 20 animals, four per evaluation timepoint, had two (2) 2 cm×2 cm full-thickness excision wounds created on their dorsum. The test and control articles were cut slightly larger than the wound periphery and applied dry to either wound following a randomized application scheme. The dressings were rehydrated by the wound fluid and sterile saline as necessary. Secondary dressings of petrolatum gauze were applied over each test and control article and changed weekly or at each evaluation timepoint. The wounds were assessed at 3, 7, 14, 28 and 42 days post-treatment. Assessments included rate and percentage wound closure (based on wound tracings), erythema, exudate and histology of explanted wound sites.

According to the results of the analysis of the percentage and rate of wound closure, the wound dressing construct treated sites demonstrated slightly faster, although not statistically significant, wound closure than the control sites. The analysis of time to complete wound closure did not find a difference between the test and control treated sites. The results of the erythema, exudate and histology analyses were equivalent for the two products. Histology showed that the wound dressing construct made from single layer ICL exhibited requisite healing characteristics over time, re-epithelialization of the wound, and resorption of the collagen materials. There was no evidence of an adverse reaction to the construct by the test subjects.

Although the foregoing invention has been described in some detail by way of illustration and example for purposes of clarity and understanding, it will be obvious to one of skill in the art that certain changes and modifications may be practiced within the scope of the appended claims.

1. A bioengineered collagen construct comprising:
a layer of purified collagenous tissue matrix derived from the tunica submucosa of a small intestine, said purified collagenous tissue matrix having been treated with an antimicrobial agent.

2. The bioengineered collagen construct of claim 1 wherein the construct is meshed, fenestrated, or perforated.

3. The bioengineered collagen construct of claim 2 wherein the mesh ratio of the mesh is 1:1.5.

4. The bioengineered collagen construct of claim 1 wherein the antimicrobial agent is selected from the group consisting of nanocrystalline silver, silver oxide, silver nitrate, silver sulfazidine, silver-imidazolate, arglaes (AgKaPO₄), Poly(hexamethylene biguanide) hydrochloride (PHMB), chlorhexadine gluconate, bis-amido polybiguanides, honey, berizalkonium chloride, triclosan (2, 4, 4′-tricloro-2′-hydroxydiphenylether), and silyl quarternary ammonium salt (octadecyl demethyl trimethoxysilyl propyl ammonium chloride).

5. The bioengineered collagen construct of claim 1 wherein the construct is crosslinked with 1-ethyl-3-(3-dimethylaminopropyl) carbodiimide hydrochloride.

6-24. (canceled)

25. A method of repairing or replacing a damaged tissue comprising the steps of:
implanting a prosthesis in a patient, said method comprising two or more superimposed, bonded layers of collagen material treated with an antimicrobial agent, wherein the prosthesis, when implanted at into a mammalian patient, undergoes controlled biodegradation occurring with adequate living cell replacement such that the implanted prosthesis is remodeled by the patient's living cells.

26. The method of claim 25, wherein the prosthesis comprises two to five sheets of processed intestinal collagen derived from the tunica submucosa of small intestine, said sheets being bonded and crosslinked together with 1-ethyl-3-(3-dimethylaminopropyl) carbodiimide hydrochloride at a concentration between 0.1 to 100 mM.

27. The method of claim 25, wherein the prosthesis comprises two to ten sheets of processed intestinal collagen derived from the tunica submucosa of small intestine, said sheets being bonded and crosslinked together with 1-ethyl-3-(3-dimethylaminopropyl) carbodiimide hydrochloride at a concentration between 0.1 to 100 mM.

28. The method of claim 25, wherein the prosthesis is a hernia repair patch, a femoral hernia repair plug, a pericardial patch, a bladder sling, a uterus sling, intracardiac patch, replacement heart valve, a vascular patch, an annular fibrosis repair plug, an annular fibrosis repair patch, a rotator cuff repair prosthesis, a dura mater repair patch, a cystocele repair device, a retrocele repair device, a vaginal vault prolapse repair sling, or a plastic surgery implant.

29. A modified intestinal collagen layer comprising an intestinal collagen layer derived from the tunica submucosa of a small intestine, said layer having been treated with an antimicrobial agent.

30. The modified intestinal collagen layer of claim 29, wherein said antimicrobial agent is selected from the group consisting of nanocrystalline silver, silver oxide, silver nitrate, silver sulfazidine, silver-imidazolate, arglaes (AgKaPO₄), Poly(hexamethylene biguanide) hydrochloride (PHMB), chlorhexadine gluconate, bis-amido polybiguanides, honey, berizalkonium chloride, triclosan (2, 4, 4′-tricloro-2′-hydroxydiphenylether), and silyl quarternary ammonium salt (octadecyl demethyl trimethoxysilyl propyl ammonium chloride).

31. The modified intestinal collagen layer of claim 30, wherein the nanocrystalline silver lacks atomic disorder.

32. (canceled)

33. A processed tissue matrix comprising a mammalian cellular tissue substantially free of non-collagenous components, said processed tissue matrix having been treated with an antimicrobial agent.

34. The processed tissue matrix of claim 33, wherein said antimicrobial agent is selected from the group consisting of nanocrystalline silver, silver oxide, silver nitrate, silver sulfazidine, silver-imidazolate, arglaes (AgKaPO₄), Poly(hexamethylene biguanide) hydrochloride (PHMB), chlorhexadine gluconate, bis-amido polybiguanides, honey, berizalkonium chloride, triclosan (2, 4, 4′-tricloro-2′-hydroxydiphenylether), and silyl quarternary ammonium salt (octadecyl demethyl trimethoxysilyl propyl ammonium chloride).

35. The processed tissue matrix of claim 34, wherein the nanocrystalline silver lacks atomic disorder.

36. (canceled)
